(19) United States
(12) Patent Application Publication
(10) Pub. No.: US 2006/0034800 A1
(43) Pub. Date: Feb. 16, 2006

(54) Method for treatment of drug addiction and for screening of pharmaceutical agents therefor

(76) Inventors: Pietro Paulo Sanna, San Diego, CA (US); George Koob, Encinitas, CA (US); Serge Ahmed, Bordeaux (FR)

Correspondence Address: Schwegman, Lundberg, Woessner & Kluth, P.A., P.O. Box 2938, Minneapolis, MN, 55402, US

(21) Appl. No.: 11/149,937

(22) Filed: Jun. 10, 2005

Related U.S. Application Data
(63) Continuation of application No. PCT/US03/39499, filed on Dec. 10, 2003.
(60) Provisional application No. 60/432,496, filed on Dec. 10, 2002.

(51) Int. Cl. A61K 38/20; A61K 38/17

(52) U.S. Cl. 424/85.2; 514/12

(57) ABSTRACT

The present invention is directed to a method for treatment of drug addiction and screening methods for identifying pharmaceutical agents that ameloriate or prevent the deleterious effects of addition. The invention is as well directed to a group of genes and a group of gene products that are up or down requested as a result of addiction.

### GOVERNMENT FUNDING

The invention described herein was developed with support from the National Institute on Drug Abuse (NIDA) under Grant Number DA 13821. The U.S. Government has certain rights in the invention.

### BACKGROUND OF THE INVENTION

A current challenge for the neuroscience of drug addiction is to understand the molecular mechanisms responsible for the development of compulsive drug use (Koob et al., 1998). Such a transition is generally associated with a pattern of escalating drug use whereby consumption increases over time and becomes more and more difficult to control. This pattern often leads to antisocial behavior, physiological addiction, physical debilitation, contraction of disease and ultimately, death.

Social scientists, behavioral scientists and biological researchers have devoted significant efforts toward ameloriating the deleterious effects of this pattern. Use of hospitalization, counseling, treatment programs and withdrawal management has been part of continuing attempts by society to minimize addiction.

Scientists have also studied the physiological changes associated with drug addiction. They have established that the body's metabolic pathways undergo significant alteration during drug addiction. In particular, these alterations make withdrawal painful and re-addiction attractive. Although the direct biochemical interactions of such opioid drugs as morphine, heroin and cocaine have been elucidated, the upstream and downstream biological effects of these interactions have not. For example, the three kinds of opioid receptors mu, delta and kappa, and the dopamine receptors are well-known as the primary receptor sites for opioid interaction. Nevertheless, how the activation of these receptors affects upstream and downstream pathways in tissues such as the central nervous system is unknown.

One of the problems facing research-scientists investigating drug addiction has been the lack of an animal model that tracks the escalating need present in humans. The known animal models typically involve plateauing consumption and effect of opioid intake. The physiological consequences of the plateau prevent the identification of genes that are up and down regulated as a result of the increasing dependency and physiological need for the opioids. In fact, few major changes in protein expression were found in the past to be related to cocaine addiction. Because of this failure, researchers have been unable to predict or correlate genetic consequences and drug dependency.

Therefore, there is a need to develop an assay to determine the up and down regulation of genes during escalating drug addiction. A further need is the identification of sets of up and down regulated genes that can be used as screens for pharmaceutical agents helpful in the treatment and/or ameloration of the causes and consequences of drug addiction. Yet another need is the identification of pharmaceutical agents that will treat the deleterious effects of addiction. A still further need is the therapeutic use of pharmaceutical agents for treatment of drug addiction where the agents do not interact with the primary opioid and dopamine receptors involved in opioid drug response.

### SUMMARY OF THE INVENTION

These and other needs are met by the present invention, which is directed to a method for treating drug addiction, especially opioid drug addiction. The invention as well is directed to a method for screening for pharmaceutical agents useful in such treatment. The invention is also directed to a set of mammalian genes that are up or down regulated during escalating drug use and to a set of corresponding gene expression products:

The treatment method according to the present invention involves administering to a patient in need of such treatment one or more pharmaceutical agents that interact with the genes which are up or down regulated during the course of escalating drug use, or that interact with the corresponding expression products, or that interact with the targets of such expression products, such as receptors. Hence, a beneficial interaction of the pharmaceutical agent is an interaction that ameliorates, blocks or prevents the abnormal up and/or down regulation of these specifically identified genes, or is an agonist, antagonist, inhibitor, activator, blocker mimic or anti-mimic of the expression product or its target.

The screening method according to the present invention involves use of an in vivo or in vitro screen to identify one or more pharmaceutical agents that interact with the expression products of genes which are up or down regulated during escalating drug use or which interact with the targets of such expression products, such as receptors.

The invention as well is directed to a set of mammalian genes and a set of their expression products that are uniquely up or down regulated during escalating opiate use. The set of genes includes those that encode certain signaling molecules or ligands, certain enzymes, certain ion channels, certain receptors, certain cytoplasmic receptor coupling proteins, certain transmembrane molecular transporters, certain ESTs and certain growth, survival, functional or structural (gsfs) proteins. In particular, these genes encode the following proteins:

A) Signaling molecules (ligands) which include insulin-like growth factor II, interleukin-3 (IL-3), interleukin-3 beta, fractalkine/chemokine CX3C motif ligand 1, platelet derived growth factor A chain, Neuroligin 3, neuron-specific protein (PEP-19), Synaptamin XI;
B) Enzymes which include catechol-O-methyltransferase, beta-andrenergic receptor kinase, Ras-related GTPase, Ras-related GTPase beta S-100, aromatic L-aminoacid decarboxylase, beta andrenergic receptor kinase, Synaptagmin III, and G-protein beta-1 subunit;
C) Ion channels which include potassium channel beta subunits, sodium channel beta 2 subunit, voltage gated potassium channel Kv3.4, Saw-related subfamily member 2, potassium channel delayed rectifier, potassium inward rectifier 10 (Kir 4.1), and calcium channel alpha 1 subunit;
D) Receptors which include AMPA receptor GluR1, Kainate receptor KA1, Peripheral benzodiazepine receptor, alpha 2-andrenergic receptor, NMDA receptor-like complex glutamate binding protein, GABBA receptor alpha 3 subunit, tumor necrosis factor receptor chain (p60), NMDA receptor subunit 2D, and non-processes neurexin1-beta mRNA;
E) Receptor coupling proteins;
F) Transporters which include vescicular inhibitory amino acid transporter and sodium dependent high affinity glutamate transporter, sodium or potassium ion transporting ATPase alpha 2 subunit,

G) ESTs which include AA799879 and AA956149, (genes);
H) Growth, survival, functional, structural proteins which include Bcl-x alpha, signal transducer and activator of transcription 3 (STAT3), Retinoblastoma protein, Nsyndecan (syndecan-3 or Neuroglycan), EST189376, Synaptotagmin VIII, Calcium ion binding protein, and Microtubule-associated protein (MAP1A).

Particularly provided are genes encoding Platelet-derived growth factor A chain; Neuroglycan; Neuroligin 3; Na⁺,K⁺-transporting ATPhase alpha 2 subunit; Na⁺,K⁺-ATPase beta 2 subunit; and NMDA receptor subunit 2.

The treatment method according to the present invention may be accomplished by administration of an effective amount any one or combination of the following:
1) an agonist or antagonist of a receptor of group D or a receptor that is a target of the foregoing group of signaling molecules, group A, including, but not limited to, NBQX, CNQX, LY300168, GYKI53655, 3-CBW, matrix metalloproteases (MM), tyrophostin AG 1024, AG1295, AG-1296 and the GABA agonist Gabapentin,
2) a mimic or anti-mimic of a signaling molecule (ligand) of foregoing group A wherein the mimic provides a similar three dimensional configuration and electronic interaction as the signaling molecule or ligand and the antinimic is the opposite, i.e., prevents binding with the corresponding target,
3) an anti-signaling molecule or anti-ligand corresponding to the foregoing group A wherein the anti-signaling or anti-ligand binds to, interferes with, or alters, such as by cleavage, the signaling molecule (ligand), including, but not limited to, matrix metalloproteases (MMP), tyiphostin, tyrphostin AG490 and batimastat,
4) an activator or inhibitor of an enzyme of foregoing group B,
5) a blocker or activator of an ion channel of foregoing group C, including, but not limited to, barium, TEA, 4AP, BDS and calphostin C,
6) an activator, inhibitor, agonist or antagonist of a receptor coupled protein of foregoing group E,
7) an activator or inhibitor of a transporter of foregoing group F,
8) an activator or inhibitor of an EST of foregoing group G,
9) an activator or inhibitor of a growth, survival, functional, structural protein of foregoing group H, including, but not limited to, tyrphostin AG490, Ghrelin, NPB/NPW, AGRP, NPY, MCH, Orexyn A/B, galanin/GALP, Beacon, beta-endorphin, dynorphin, GHRF, alpha-MSH, CART, PYY3-36, NPB, CRF, urocortin II, III, GLP-I, oxytocin, neurotensin, CCK, GRP, bombinakinin-GAP, neuromedin, POMC, ADM, somatostatin, TRH, and CGRP.

The pharmaceutical agent effective for treatment according to the invention may be administered as a pharmaceutical composition of a pharmaceutical agent and a pharmaceutical carrier. The carrier is chosen according to the dictates of the route of administration.

The method for screening according to the invention may be accomplished by in vivo or in vitro techniques. The in vivo technique involves use of an animal model and either a historical or current positive control wherein the test animals are treated with an increasing dosage of addicting drug and before, simultaneous with, or after beginning the addicting drug administration, are given the potential pharmaceutical agent. mRNAs from specified brain sections of the test animals can be obtained sequentially and screened in a multi-well assay to determine up and down regulation of the genes mentioned above. A lessening of the up and/or down regulation of one or more of these genes relative to the historical or current positive control indicates that the potential pharmaceutical agent will be useful in the treatment of drug addiction.

The method for screening according to the invention may also be accomplished by an in vitro technique. Cells may be contacted with a potential pharmaceutical agent and mRNA may be extracted from the cells. The mRNAs can be screened to determine if the potential pharmaceutical agent caused an increase or decrease in the expression of the gene products described herein as associated with drug addiction. Gene expression may also be determined through use of other known biological assays that include radioimmunoassay, ELISA, southern blot, northern blot, enzymatic activity and the like to establish whether or not appropriate activity is present.

### BRIEF DESCRIPTION OF THE DRAWINGS

FIG. 1.a) Escalation in intravenous cocaine consumption in rats. Mean (±s.e.m.) number of intravenous cocaine self-injections obtained during the first hour of each daily session of cocaine self-administration. (* different from ShA rats, p<0.05, tests of simple main effects after appropriate two-way analyses of variance).

FIG. 1.b) Total number of probe sets per brain region that significantly change by more than 1.8-fold in LgA (long access) rats compared to control levels measured in drug-naive rats. (c) Fraction of total probe sets that significantly change in LgA rats compared to both ShA (short access) and drug-naive rats (ES genes). Abbreviations: VTA, ventral tegmental area; LH, lateral hypothalamic area; AMG, amygaloid complex; ACC, nucleus accumbens; SEP, septal area; PFC, medial prefrontal cortex.

FIG. 2. Correlation between changes in gene expression levels in rats with differential access to intravenous cocaine self-administration (see Methods). In both groups, the expression level corresponding to each probe set was normalized to the control level measured in drug-naive rats (see Methods for details). Normalized values range from 0 to 1, with 0.5 corresponding to no change from the control level. The central square in each graph contains all probe sets that do not change by more than 1.8-fold in both ShA rats and LgA rats (see Methods for details). Each point represent a single gene (over 1300 probe sets) and each graph represents a different reward-related region of the brain (6 in total).

### DETAILED DESCRIPTION OF THE INVENTION

The present invention is based upon an animal model for drug addiction that more accurately tracks the course of drug addiction in man. While traditional models limit access to the addicting drug, this model enables ever-increasing dosing if desired by the test animal. In this model, drug intake gradually escalates over time when daily access to the drug is increased to 6 or more hours (Ahmed et al., 2000; Ahmed and Koob, 1998). Using this model, genes specifically associated with drug addiction in selected reward-related-brain regions have been identified.

The opiate, cocaine, was the drug of choice used in the study. This drug displays a typical opioid addiction pattern and will predict the behavior and physicological reaction of the group of opioid drugs. It is known to interact with the opioid and dopamine receptors of the central nervous system of mammals. However, the methods of the invention may also be used in association with other addictive substances.

Thus, the present invention specifically investigates escalation of cocaine intake, which a) is a superior model for drug addiction and b) selects from the large number of altered transcripts in the transcriptional profilings only those mRNAs and gene products which themselves, or the ligands thereof, could be used to treat human drug addiction.

According to the invention, the raw experimental evidence shows that a large number of genes are responsive to cocaine self-administration (self-administration-associated genes, SA genes). However, when the results using the traditional model and the new model of administration are compared, only a small fraction of those genes changed their expression specifically in association with escalation of cocaine intake (escalation-associated genes, ES genes). Of all the brain regions examined, the lateral hypothalamus area was the most genetically responsive. The pattern of ES genes observed within this area indicates that compulsive drug use is associated with a profound remodeling of lateral hypothalamic intrinsic circuitry involving glutamatergic neurotransmission. Many of the ES genes identified are also expressed during development and/or are involved in neural plastic processes in the adult brain, such as neurogenesis, synaptogenesis, regulation of synaptic strength and responses to neurotoxic stress. It is believed that these results indicate that brain reward pathways undergo a large-scale reorganization, both structurally and functionally, during the transition to drug addiction. These neuroadaptive changes contribute to the chronic deficit in reward function recently reported after cocaine intake escalation (Ahmed et al., 2002).

Accordingly, the invention concerns the identification of gene targets in the escalating addiction animal model that have already interacted, or will interact, with the addicting drug. Identification of these up and down regulated genes of the animal model and their correlation with corresponding human genes predicts physiological changes occurring in human addiction. The identification also enables significant advances in treatment of addiction.

According to the invention, the identified gene targets include the following.
A) Genes encoding signaling molecules that include Insulin-like growth factor II, interleukin-3 (13), interleukin-3 beta, fractadkine/chemokine Cx3 C motif ligand, neuroligin 3, PDGF, neuron-specific protein (PEP-19), and Synaptamin XI;
   These signaling molecules, and the agonists and antagonists for their corresponding receptors, as well as mimics and antiminics may be used to treat drug addiction.
B) Genes encoding specific enzymes including Catechol-O-methyltransferase (COMT), Synaptagmin m, Beta-adrenergic receptor kinase, Ras-related GTPhase (Rab3), Ras-related GTPase beta S-100, aromatic L-amino acid decarboxylase (DOPA decarboxylase) and G-protein beta-1 subunit (rGbeta1);
   These enzymes and their activators and inhibitors may be used to treat drug addiction.
C) Genes encoding ion channels including K+ channel beta subunits (Kv1-type), Na+ channel beta 2 subunit (Scn2b), voltage gated K+ channel Kv3.4, Shaw-related subfamily member 2 (Kcnc2), K+ channel delayed rectifier (RCK2), K+ inward rectifier 10 (Kir 4.1), and Ca++ channel alpha 1 subunit (Cacna1);
   These ion channel proteins and their blockers and activators may be used to treat drug addiction. It should be noted that, for instance, Novartis has an inhibitor of COMT Comtan (Entacapone) used for the treatment of Parkinson.
D) Genes encoding receptors, which overlap but are not coterminus with the receptors mentioned in A, and which include AMPA receptor GluR1, Kainate receptor KA1, Peripheral benzodiazepine receptor (PKBS), alpha 2-Adrenergic receptor (RG20), NMDA receptor subunit 2, NMDA receptor-like complex glutamate binding protein (GBP), non-process neurexin 1-beta mRNA, GABAA receptor alpha 3 subunit, MAP1A, and NMDA 2D receptor;
   These receptors and their agonists and antagonists may be used to treat drug addiction.
E) Genes encoding receptor-coupled proteins;
   These receptor coupling proteins and their activators, inhibitors, agonists and antagonists may be used to treat drug addiction.
F) Genes encoding transporters exemplified by the vescicular inhibitory amino acid transporter (5VLIAT), Na+ dependent high affinity glutamate transporter (GLT-1A), and sodium ATPase isoform, potassium ATPase isoform;
   These transporters and their activators and inhibitors may be used to treat drug addiction.
G) ESTs exemplified by AA799879 and AA956149;
   The gene products of these EST's and ligands for such gene products may be used to treat drug addiction.
H) Genes encoding growth, survival, functional, structural (gsfs) proteins exemplified by Bcl-x alpha, signal transducer and activation of transcription 3, Retinoblastoma protein, Nsyndecan (syndecan-3 or Neuroglycan), EST 189376, Synaptotagmin VIII, calcium ion binding protein, and microtubule-associated protein (MAP1A);
   These gsfs proteins and their activators and inhibitors may be used to treat a drug addition.
      Preparation of Proteins, Oligopeptides and Peptides of A Through G

The gene expression products of A through G (see Table I) above may be proteins, shorter oligopeptides or short peptides. All may be generally characterized as polypeptides. Consequently, that term is used in this section as a synonym for proteins, oligopeptides and peptides. The polypeptides can be expressed in vivo through use of prokaryotic or eukaryotic expression systems. Many such expressions systems are known in the art and are commercially available. (Clontech, Palo Alto, Calif.; Stratagene, La Jolla, Calif.). Examples of such systems include, but are not limited to, the T7-expression system in prokaryotes and the bacculovirus expression system in eukaryotes. Such expression systems are well known and have been described. Sambrook and Russell, Molecular Cloning, A Laboratory Manual, Cold Spring Harbor Laboratory Press, Cold Spring Harbor, N.Y., 2001.

Polypeptides can also be synthesized in vitro, e.g., by the solid phase peptide synthetic method or by in vitro transcription/translation systems. The synthesis products may be fusion polypeptides, i.e., the polypeptide comprises the polypeptide variant or derivative according to the invention and another peptide or polypeptide, e.g., a His, HA or EE tag. Mimics and antimimics may also be synthesized in vivo or in vitro. Mimics are generally molecules that mimic the structure of a ligand that is bound by a receptor. Thus, mimics are generally used to bind and stimulate a receptor. Antimimcs are generally molecules that mimic the structure of a ligand bound by a receptor that decrease the activity of a receptor upon binding. Methods to synthesize polypeptides are described, for example, in U.S. Pat. Nos. 5,595,887; 5,116,750; 5,168,049 and 5,053,133; Olson et al., Peptides, 2, 301, 307 (1988). The solid phase peptide synthetic method is an established and widely used method, which is described in the following references: Stewart et al., Solid Phase Peptide Synthesis W.H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Method for Screening

The invention includes a method to determine if a pharmaceutical agent is able to act as an agonist, antagonist, inhibitor, blocker, activator, mimic or antimimic of a gene product or, in the case of a signaling molecule, the associated receptor. In this instance, a pharmaceutical agent may be a peptide, oligopeptide or organic small molecule of any kind. The method can be used to determine if the pharmaceutical agent increases, decreases, activates, blocks, inhibits, mimics or prevents the action of the gene product. The method may be conducted under in vivo or in vitro conditions.

Potential pharmaceutical agents can be screened in vivo for their ability to decrease drug addition. This may be done by first offered an animal long-term access to an addicting drug such that the animal exhibits an altered mRNA expression profile when compared to animals offered short-term access to the addicting drug and non-exposed control animals. Next, one or more potential pharmaceutical agents can be administered to the experimental animal offered long-term access to the addictive drug. The experimental animal can then be sacrificed and mRNAs can be extracted from the brain of the experimental animal and such that the expression levels in individual genes (such as those described in Table I) may be determined or compared to a control. Methods to determine the expression level of mRNA are known in the art and include, Northern blotting, use of a nucleic acid array or chip, and the like. The expression level of mRNAs extracted from the experimental animal can be compared to those from animals offered short-term access to the addicting drug and to non-exposed control animals. Increased expression in response to the potential pharmaceutical agent of an mRNA that is decreased in an addicted animal indicates that the potential pharmaceutical agent acts to ameliorate addiction. Also, decreased expression in response to the potential pharmaceutical agent of an mRNA that is increased in an addicted animal indicates that the potential pharmaceutical agent acts to ameliorate addiction.

In vitro methods may also be used to screen a potential pharmaceutical agent for the ability to ameliorate drug addiction. For example, an in vitro method can involve contacting a pharmaceutical agent with a cell that expresses a gene encoding a product included within groups A through H and/or Tables 1 and 2. Altered expression of an mRNA in response to the potential pharmaceutical agent may be determined by extracting mRNA from the contacted cell and comparing expression of a selected mRNA to that in a control cell that was not contacted with the potential pharmaceutical agent.

The methods of the invention may be used under nearly any conditions wherein a potential pharmaceutical agent can come into contact with a cell. For example, the cells in contact with the potential pharmaceutical agent may be grown on plates, grown in liquid culture, grown in monolayers, or be located in vivo within the body of an organism. Large or small numbers of cells may be used within the methods of the invention. Methods to culture cells are well known in the art and are disclosed herein. Parameters, such as the temperature, time, growth media, pH, and atmosphere used during incubation of the cells with the potential pharmaceutical agent may be adjusted to accommodate specific cell types according to well known procedures.

The methods of the invention also include the use of detectable labels that can be used to detect binding events, such as those occurring during the binding of a ligand, such as a signaling molecule, by a receptor (such as those disclosed in Table I). In one example, a signaling molecule encoded by an mRNA having expression that is increased or decreased in response to drug addiction may be labeled with a detectable label. A potential pharmaceutical agent can then be added to a mixture containing a cell that expresses a receptor to the labeled signaling molecule and incubated under conditions wherein the receptor can bind to the ligand. The incubation mixture can then be washed and the amount of labeled ligand bound to the cell can be determined through detection of the detectable label. Such methods allow potential pharmaceutical agents to be screened for their ability to increase or decrease binding of a ligand by a receptor and ameliorate drug addiction.

Numerous detectable labels are known in the art and include, fluorescent proteins, enzymes, antigenic tags, and the like. Such labeled ligands may be expressed within a cell from an exogenous nucleic acid segment. For example, a vector may encode a ligand that is linked to a fluorescent protein and used to express the labeled ligand in a cell. A nucleic acid segment introduced into a cell may encode one or more detectable labels. In addition, a nucleic acid segment introduced into a cell may encode gene products other than detectable labels. Recombinant nucleic acid techniques, cloning vectors, and cellular transformation methods are well known in the art and have been described. Sambrook et al., Molecular Cloning: A Laboratory Manual, 3rd edition, Cold Spring Harbor Press, Cold Spring Harbor, N.Y. (2001).

Numerous types of cells may be utilized within the methods of the invention. Such cells can be engineered to allow expression of a desired nucleic acid segment, such as a detectable label. Naturally occurring and immortalized cells may be used within the invention. Genetically modified cells may also be used within the methods of the invention. For example, a cell may be transformed with a nucleic acid construct that directs the expression of a gene product of A through G (as described in Table I) not normally expressed by the cell. Accordingly, genetically modified cells can be constructed to express selected receptors for the potential pharmaceutical agent. Thus, genetically modified cells may be matched with potential pharmaceutical agents and used within the methods of the invention. Such combinations allow one of skill in the art to produce genetically modified cells and gene products that may be used to identify potential pharmaceutical agents.

Use of the in vitro methods of the invention to screen potential pharmaceutical agents may provide any number of results including blockage, activation, inhibition, increasing, decreasing, augmenting and catalyzing gene product function. Use of a single screen will also be effective for identification of potential pharmaceutical agents.

Qualitative and quantitative assays may be conducted. Both will determine whether the interaction sought has occurred. Quantitative assays will enable identification of an increase, decrease or augmentation of gene product function.

The typical assay will be based upon the function of the gene product involved. For signaling molecules, the appropriate receptor will also be present. This receptor may include its natural enzyme domain to convert the detectable label or may be re-engineered to convert the detectable label. Alternatively, an antibody assay for the bound and/or unbound forms of the signaling molecule may be used. In such an assay, the detection of the detectable label produced by the receptor or through the antibody assay will indicate activity of the candidate.

For enzymes, the enzymatic activity may be employed in combination with a detectable label to determine potential pharmaceutical agent interaction. Incorporation of a detectable label into a substrate for the enzyme where the detectable label is released upon enzymatic activity will provide an appropriate in vitro assay. The potential pharmaceutical agent activity for activation, inhibition and the like of the enzyme can then be determined by measuring the quantity of detectable label produced.

For ion channels, incorporation into an artificial membrane and determination of the ability of the membrane to pass the appropriate ions may be employed as an appropriate in vitro assay. This assay mimics an in vivo assay using the degree of ion passage through an appropriate cellular membrane.

Receptors and receptor-coupled proteins may be assayed as described above for signaling molecules. In these instances, the downstream action of an enzymatic domain or triggered enzyme may be employed to appropriate advantage for assaying these gene products according to the invention.

Transporter molecules may be assayed for their ability to transport their corresponding substrate molecule which has been modified with a detectable label. An intact cellular membrane or artificial membrane may be employed as the functional system in which the transporter molecule operates. Assay of the detectable label delivered, or not delivered across the membrane by the transporter molecule will identify potential pharmaceutical agents interacting with these molecules.

Many methods may be used to detect the detectable label. Chemiluminescence may be used to detect the detectable label. Briefly, the detectable label can be contacted with a substrate that is acted upon by the detectable label to produce a signal that may be detected with a luminometer. For example, the following detectable labels and their substrates are provided as examples that may be used for chemiluminescent detection of cellular invasion: alkaline phosphatase with AMPPD; β-galactosidase with AMPGD; horseradish peroxidase with lininol+perborate+4-iodophenol; and xanthine oxidase with luminol+Fe EDTA (Harlow et al., Antibodies: A Laboratory Manual, page 319 (Cold Spring Harbor Pub. 1988)). Bioluminescence may be used in an analogous manner as chemiluminescence to detect a detectable label. Fluorescence may be used to detect a fluorescent protein that is produced, transported, converted or expressed as a detectable label. For example, green fluorescent protein may be the result of any of the foregoing in vivo or in vitro assays and may be detected with a fluorimeter, a fluorescent plate reader, or a fluorescent microscope. Ultraviolet or visible light may be used to detect the presence of a detectable label produced in an assay according to the invention. Such detection methods are known in the art and are disclosed herein.

Agonists, Antagonists, Activators, Blockers, Inhibitors, Ligands, Anti-ligands, Anti-Signaling Molecules, Mimics, and Anti-Mimics (See 1-9 Above) of Proteins A Through H

Secretases (sheddases) can be useful as therapeutic targets in cocaine addiction. Several proteins have been identified as members of a diverse range of membrane proteins that also occur as soluble forms derived from the membrane form by proteolysis. Protease cleavage regulates the activity of these proteins. Inhibition of protease cleavage of the ectodomains of these proteins could interfere with the biological process induced by the escalation of cocaine addiction. Proteolytic cleavage of the ectodomains of these membrane proteins is carried out by a group of enzymes referred to collectively as ‘secretases’ or ‘sheddases’. The majority of secretases are matrix metalloproteases (MMP). These shed membrane proteins identified as being induced during the escalation of cocaine addiction include, but are not limited to, syndecan 3, fractalkine, and TNF receptor (p60), which ligand TNF-alpha is also regulated by proteolytic cleavage of its ectodomain. Additionally, PDGF-A was found to be increased and the PDGF receptor ectodomain is also released by protease cleaveage. The notion that dysregulation of the secreatase system could be induced by the escalation of cocaine addiction is also supported by the observation that tissue inhibitor metalloproteinase 3 (TIMP-3) was found to be increased by escalation of cocaine intake. TIMP-3 has been shown to inhibit syndecan 3 cleavage and, like syndecan, it is increased by food deprivation (Reizes O., 2003). TIMP-3 preferentially inhibits MMP-1, -3, -7, -13 and the TNF-alpha-converting enzyme (TACE) (Stamenkovic, 2003), although inhibitory activities of different TIMPs towards different MMPs are not particularly selective. Notably, PDGF-A has been shown to upregulated MMPs in some tissues (Robbins 1999). Many proteins released by ectodomain cleavage have been previously disclosed to be involved in pathophysiological processes such as neurodegeneration, apoptosis, oncogenesis and inflammation, and therefore secretases have received great attention as possible therapeutic targets. In addition, another tissue protease system, the tissue plasminogen activator (t-PA) was found to be induced. T-PA has been implicated in synaptic plasticity (discussed in Nicholas, 2003) and potentiates NMDA-receptor function (Nicole, 2001).

TNF receptor (p60): The observed decrease in TNF receptor (p60) may reflect induction of TNF-alpha Shedding of membrane-bound pro-TNF-alpha is thought to be largely due to TNF-alpha-converting enzyme (TACE), therefore TACE inhibitors could be beneficial. Large collections of MMP inhibitors, including TACE inhibitors are being developed by several companies (reviewed in Hooper 1997). (For example, see http://www.uspto.gov/ for patent and patent publications that are assigned to Pfizer (Letavic et al. 2003), Wyeth Research (Levin et al. 2001a, 2001b, 2002 and 2003; Zask et al. 2003; Nelson et al. 2003; Chen 2002), Glaxo Wellcome (Conway et al. 2001), Immunex Corporation (Mullberg, 1995) and Bristol-Myers (Duan et al 2002), such patents and patent publications are hereby incorporated by referenced).

Fractalkine: Fractalkine acts as a neuron- or endothelial-derived intercellular signaling molecule to attract proinflammatory cells after excitotoxic injury, such events are amplified by fractalkine cleavage, which is promoted by TNF-alpha and other cytolines. Blocking fractalkine cleavage with the secretase inhibitor Batimastat (AKA BB94, Glaxo-SmithKline) inhibits these events (Chapman, 2000).

PDGF-A and the PDGF-alpha receptor (PDGFR-alpha) are present in various neuronal populations in the adult CNS. PDGF receptor inhibitors have been established as antitumor drugs, including several tyrphostin compounds like AG1295, AG-1296 (Levitzki A 1999, Lipson 1998).

Syndecan 3: As discussed above, the activity of syndecan can be modulated by secretases. During food deprivation, TIMP-3 is induced, resulting in inhibition of a sheddase or matrix metalloprotease, leading to an increase in cell surface expression of syndecan-3. Similarly, it was observed that both Syndecan 3 and TIMP-3 were induced in cocaine escalating rats (Reizes, 2003). Exogenous matrix metalloprotease inhibitor or increased TIMP-3 expression results in increased syndecan-3 expression and increased food intake (Reizes, 2003). Syndecan 3 has been shown to increase the action of the orexigenic peptide AGRP which acts as an endogenous competitive antagonist of alpha-melanocyte-stimulating hormone (alpha-MSH) at the melanocortin-3 and 4 receptors. This analogy between the systems controlling food intake and drug abuse suggests that drugs being developed to treat obesity by acting on orexigenic (Ghrelin, NPB/NPW, AGRP, NPY, MCH, Orexyn A/B, galanin/GALP, Beacon, beta-endorphin, dynorphin, GHRF) and anorexigenic (alpha-MSH, CART, PYY3-36, NPB, CRF, urocortin II, III, GLP-I, oxytocin, neurotensin, CCK, GRP, bombinakinin-GAP, neuromedin, POMC, ADM, somatostatin, TRH, CGRP) peptide systems could also be beneficial in drug abuse. Prior to Applicants' invention, AGRP, the peptide most likely to be directly regulated by syndecan has not previously been associated with drugs of abuse, including cocaine. However, Lindblom et al (May 2002) suggest that the AA strain of alcohol preferring rats have a high ratio of POMC/AGRP expression, and that this observation is accompanied by differences in MC3 receptor levels. Also, the non-selective MC-receptor agonist MTII caused a reduction in ethanol intake and ethanol preference in AA rats (Ploj K 2002 October). Earlier work had implicated the melanocortins in opiate addiction (Alvaro 1997) and recently in the effects of cocaine (Alvaro 2003), which appear to be opposite to those of opiates (morphine down-regulates the expression of MC4-R in striatum and periaqueductal gray while cocaine up-regulates MC4-R mRNA expression in the striatum and hippocampus (Alvaro 2003)). However, AGRP had not been previously associated with cocaine addiction and nor have there been any studies on the regulation of these systems in the hypothalamus where changes in syndecan regulation where demonstrated herein.

Tissue plasminogen activator (t-PA): t-PA was increased in the lateral hypothalamus of cocaine escalating rats, while plasminogen activator inhibitor 2 (PAI-2) was slightly decreased. Plasminogen activators convert plasminogen to the active protease plasmin and have been previously implicated in brain plasticity and in toxicity inflicted in hippocampal pyramidal neurons by kainate (Sharon 2002) and hypoxia (Hosomi 2001). Additionally, t-PA potentiates signaling by glutamatergic receptors by cleaving the NR1 subunit of the NMDA receptor resulting in a 37% increase in NMDA-receptor function. These results were confirmed in vivo by the intrastriatal injection of recombinant-PA, which potentiated the excitotoxic lesions induced by NMDA (Nicole 2001). A role for t-PA in neural plasticity is supported by observations that t-PA overexpression improves water maze performance, additionally long-term potentiation (LTP) induction in hippocampal slices is associated with an increase in tPA expression, and inhibitors of tPA activity impair late-phase LTP in hippocampal slices (discussed in Nicholas, 2003). A synthetic tPA/plasmin inhibitor is called tPA-stop (America Diagnostica Inc. #544).

IGF: Both pharmacological inhibitor and gene therapy approaches are being developed to inhibit the IGF system as antitumor strategies. A pharmacological example is Tyrphostin AG 1024 (Parrizas et al 1997) and an example of gene therapy strategy is disclose in Johnson et al. (1994).

Stat 3: The JAK family-specific inhibitor, tyrphostin AG490, markedly inhibits Stat3 activation (Toyonaga, 2003; Zhang 2000).

IL-3: Mice transgenic for IL-3 under the control of the GFP promoter develop progressive motor disease at approximately 5 months. Lesions identified after disease onset showed activation of microglia, astroglial proliferation with phagocytosis of lipids, and immigration of macrophages and mast cells into neural parenchyma. Therefore overexpression of IL-3 in cocaine escalation could contribute to microglia activation and promotion of inflammation. Agents that inhibit microglia proliferation include, but are not limited to, the aforementioned inhibitors of the shedding of fractalkine and could be beneficial by countering the action of IL-3. The JAK family-specific inhibitor, tyrphostin AG490 that inhibits Stat3 activation (Toyonaga, 2003; Zhang 2000) also blocks most effects of IL-3 (Si and Collins 2002).

Kv3.4 blockers: tetraethylammonium (TEA), 4 aminopyridine (4AP), BDS,

Kir4.1 blockers: barium.

K+ channel beta subunit inhibitor: calphostin C.

Periferal Benzodiazepine receptor (PKBS): PKBS has been known to have many functions such as a role in cell proliferation, cell differentiation, steroidogenesis, calcium flow, cellular respiration, cellular immunity, malignancy, and apoptosis. Its expression in the brain mostly reflects astrocytes and microglia activation (Versijpt, 2003). Ligands include, in order of affinity: PK11195=Ro5-4864>FGIN-1-27>triazolam=diazepam>beta-pro-pyl-beta-carbolhne-3-carboxylate=clonazepam>lorazepam=flurazepam>>chlordiazepoxide=clorazepate. Treatment with peripheral (Ro5-4864) and mixed (diazepam), but not central (clonazepam), benzodiazepine receptor ligands blocked certain aspects of microglia activation (Lokensgard 1998, 2001). PK11195 is used for visualization of neuroinflammation in vivo (Cagnin A, 2002).

GluR1: AMPA receptor inhibitors NBQX, CNQX, LY300168 GYKI53655.

Kainate receptor antagonists: CNQX at high dose, 3-CBW.

GABAA alpha3 subunit: the GABA agonist Gabapentin.

Pharmaceutical Compositions

According to the invention, the gene products and the related agonists, antagonists, activators, blockers, inhibitors, ligands, mimics, antimimics of A through H above may be chemically configured as proteins, oligopeptides and small organic molecules. Together, these compounds will be discussed in this section as proteins and related molecules. The proteins and related molecules of the invention may be formulated into a variety of acceptable compositions. Such pharmaceutical compositions can be administered to a mammalian host, such as a human patient, in a variety of forms adapted to the chosen route of administration, i.e., orally or parenterally, by intravenous, intramuscular, topical or subcutaneous routes.

In cases where the proteins and related molecules are sufficiently basic or acidic to form stable nontoxic acid or base salts, administration of such proteins and related molecules, as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made.

Thus, the present proteins and related molecules, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the proteins and related molecules, may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of oxidants and oxygen scavengers in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The proteins and related molecules may also be administered intravenously or intraperitoneally by infusion or injection. Solutions of the proteins and related molecules may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

The pharmaceutical dosage forms suitable for injection or infusion can include sterile aqueous solutions or dispersions or sterile powders comprising the proteins and related molecules that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the proteins and related molecules in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the oxidants and oxygen scavengers plus any additional desired ingredient present in the previously sterile-filtered solutions.

For topical administration, the proteins and related molecules may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the proteins and related molecules of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the proteins and related molecules of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the proteins and related molecules or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram-body weight of the recipient per day, preferably in the range of 6 to 90 mg/kg/day, most preferably in the range of 15 to 60 mg/kg/day.

The proteins and related molecules are conveniently administered in unit dosage form; for example, containing 5 to 1000 mg, conveniently 10 to 750 mg, most conveniently, 50 to 500 mg of active ingredient per unit dosage form.

Ideally, the proteins and related molecules should be administered to achieve peak plasma concentrations of the proteins and related molecules of from about 0.005 to about 75 μM, preferably, about 0.01 to 50 μM, most preferably, about 0.1 to about 30 μM. This may be achieved, for example, by the intravenous injection of a 0.05 to 5% solution of the proteins and related molecules, optionally in saline, or orally administered as a bolus containing about 1-100 mg of the proteins and related molecules. Desirable blood levels may be maintained by continuous infusion to provide about 0.01-5.0 mg/kg/hr or by intermittent infusions containing about 0.4-15 mg/kg of the proteins and related molecules.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The therapeutic compositions of this invention, proteins and related molecules that include both engineered proteins and related molecules and other molecules containing additional reductive centers as described herein for promoting proteins and related molecules activity, are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for various types of applications depend on the route of administration. Suitable regimes for administration are also variable, but are typified by an initial administration followed by repeated doses at intervals to result in the desired outcome of the therapeutic treatment.

Therapeutic compositions of the present invention contain a pharmaceutically acceptable carrier together with the proteins and related molecules. In a preferred embodiment, the therapeutic composition is not immunogenic when administered to a mammal or human patient for therapeutic purposes.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic compositions of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Pharmaceutically acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

The invention is further described in detail by reference to the non-limiting examples that follow. While the invention has been described in detail with reference to certain preferred embodiments thereof, it will be understood that modifications and variations are within the spirit and scope of that which is described and claimed.

Exemplary Protocol

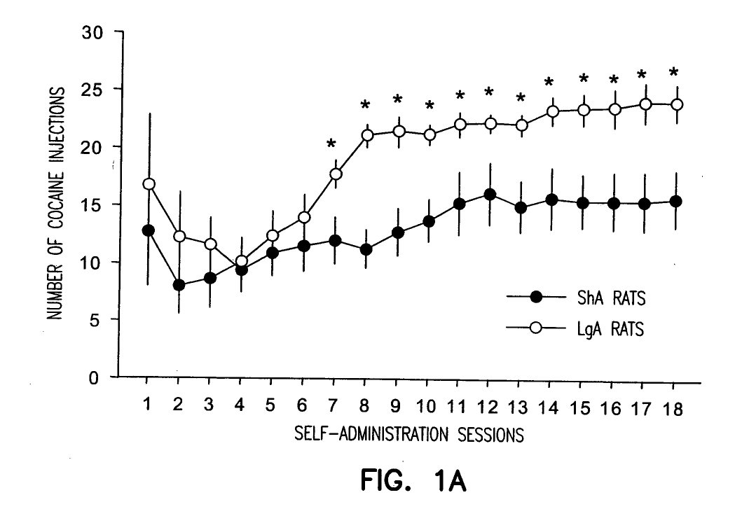

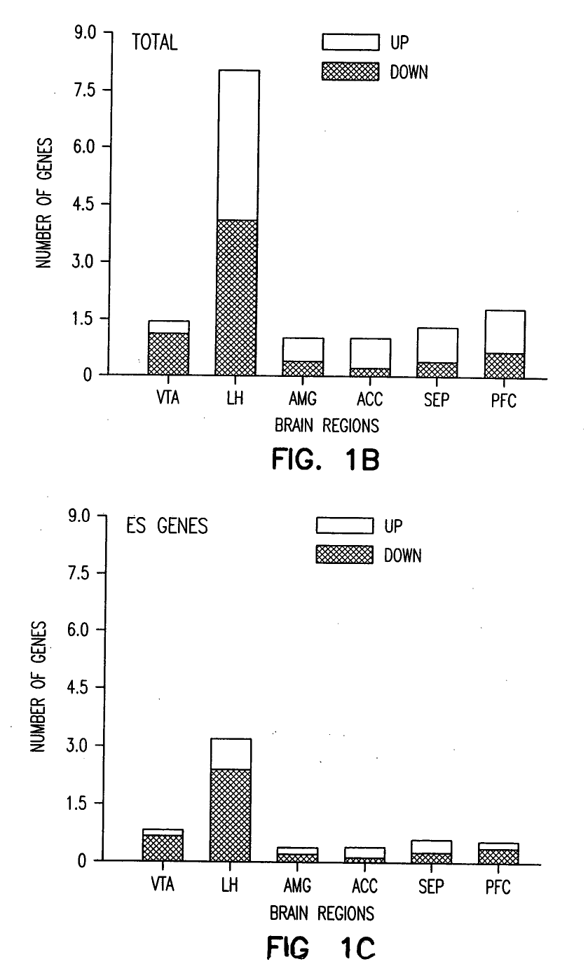

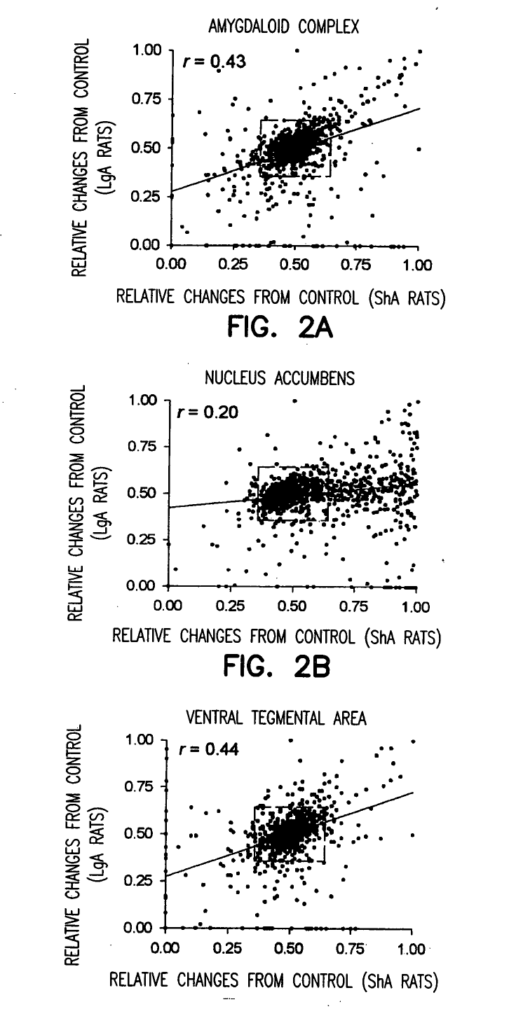

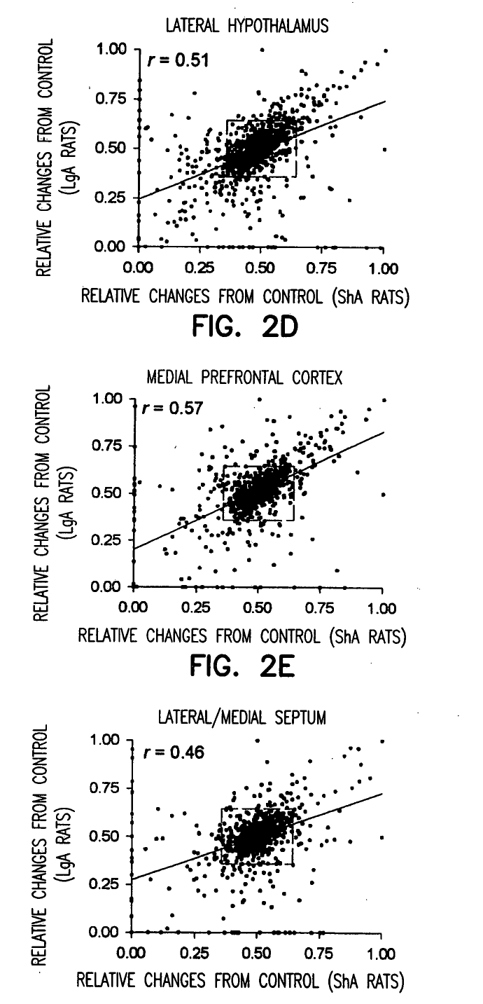

In rats allowed to self-administer cocaine, the duration of access dramatically influenced cocaine intake. Within 18 days, the first hour of cocaine intake in LgA rats rose to a level almost two times greater than that observed in ShA rats, which, as expected, remained stable over time (FIG. 1). Total intake in LgA rats also increased over the same period of time from an initial average of 48 to 126 cocaine injections. Forty-eight hours after the last self-administration session, all animals were sacrificed to obtain tissue samples from 6 reward-related brain regions: ventral tegmental area (VTA), lateral hypothalamus (LH), amygdala (AMG), nucleus accumbens (ACC), septum (SEP) and prefrontal cortex (PFC). Gene expression profiling was then performed for each dissected brain region using the Affymetrix Rat Neurobiology Array. This array consists of over 1300 probe sets representing all known neurotransmitter receptors, transporters, synthetic and metabolic enzymes, signal transduction proteins, as well as other brain-specific transcripts. Relative variations from control levels in ShA and LgA probe sets are plotted together in FIG. 2. Regression analysis showed a positive correlation gene expression changes between cocaine-exposed groups (all r values were above 0.43, p<0.01); this correlation was the lowest in the nucleus accumbens (r=0.20, p<0.01). Thus, regardless of the brain region considered, the majority of genes whose expression levels are affected after exposure to cocaine self-administration were not differentially affected by the pattern of cocaine intake (stable/moderate in ShA rats vs. escalating/excessive in LgA rats).

As shown in Tables 1 and 2, ES genes can be classed in four functional categories: 1) genes coding for proteins involved in the regulation of neuronal growth, survival and functional and structural plasticity; 2) genes coding for proteins involved in the regulation of membrane potential such as ion pumps and channels; and 3) neurotransmitter receptors, synthetic and metabolic enzymes and transducers; and 4) genes involved in the neurotransmitter release machinery. For convenience, the tabular chart presenting this information has been divided into Tables 1 and 2. The graphs of Table 2 correlate with the charted information of Table 1 as indicated by the gene listings. Consequently, the graphs of Table 2 align with the rows of Table 1 according to the gene names.

Table 3 presents the results of hybridization of the lateral hypothalamus with Affymetrix chip: RAE-230A expression array (the last 3 were obtained with the dChip analysis software that is logarithmic and therefore significance is obtained with lower fold changes). The columns are: probe set (Affymetrix id of probes on the chip); accession number (general identifier for the gene sequence from which the probe is derived); FC C/A (fold change between condition C (cocaine escalating rats) and A (control)); FC C/B (fold change between condition C (cocaine escalating rats) and B (cocaine NON escalating rats); Gene (name of the gene); and Software used to generate the fold change value (MAS 5.0 or dChip 1.3).

Table 4 discloses a large number of candidate genes that appear to be associated with the development of the escalation of cocoaine intake/addiction. The data presented in Table 4 is the product of repeated analysis with various algorithms. The columns are: probe set (Affymetrix id of probes on the chip); accession number (general identifier for the gene sequence from which the probe is derived); FC C/A (fold change between condition C (cocaine escalating rats) and A (control)); FC C/B (fold change between condition C (cocaine escalating rats) and B (cocaine NON escalating rats); and title (name of the gene).

The lateral hypothalamus was the brain structure that revealed the greatest changes in gene expression. Several genes involved in structural plasticity changed with cocaine escalation in this area Examples of such genes are the alpha2 and beta2 isoforms of Na+, K+-ATPase isoforms, which have been shown to be induced in Schwann cells during peripheral nerve regeneration (Kawai et al., 1997); the proteoglycan N-syndecan (syndecan-3 or neuroglycan), which is transiently expressed on growing axons during development and binds heparin-binding growth factors with neurite-promoting activity (Bandtlow and Zimmermann, 2000); Neuroligin 3, a member of a family of synaptically associated adhesion molecules, which has been implicated in synaptogenesis (Cantallops and Cline, 2000), was also found to be induced in the LH. Increased transcription of the trophic factor PDGF, its transducer STAT3, and the anti-apoptotic factor Bcl-xalpha—whose transcription is regulated by PDGF and STAT3 (Huang et al., 2000; Stephanou et al., 2000)—was also seen in the LH of LgA rats. This coordinate pattern of gene expression changes indicates a response to a pro-apoptotic insult in hypothalamic cells of animals that have developed escalated levels of drug intake. The transcript for the chemokine fractalkine was also upregulated in the LH of escalating rats. Fractalkine is a chemokine predominantly expressed in the brain, which is believed to be part of a mechanism response to excitotoxic neuronal injuries (Chapman et al., 2000). Both fractalkine and PDGF reduce glutamate neurotransmission and their activation could be a response to chronic activation of glutamate-mediated excitatory neurotransmission (Chapman et al., 2000; Sims et al., 2000).

Changes in the expression of selected glutamate receptors were also observed. In particular, in the lateral hypothalamus, expression of the AMPA receptor subunit 1 (GluR1) was decreased and expression of N-methyl-D-Aspartate receptor subunits 2D (NR2D) was increased in rats that have developed escalated levels of cocaine intake. Expression of GluR1 has been found to be increased in the VTA following repeated administration of morphine and cocaine (Carlezon et al., 1997) and viral mediated overexpression of this receptor in the VTA induces sensitization to morphine (Carlezon et al., 2001). Interestingly, however, intacranial self stimulation in the LH has been shown to decrease GluR1 expression in the VTA (Carlezon et al., 2001). GluR1 expression was not significantly increased in the VTA in both LgA and ShA rats (not shown). GluR2 was significantly decreased in both LgA and ShA rats in the LH (not shown). The messenger for kainate-type glutamate receptor 1 (KA) was also decreased in escalating rats. The down-regulation of GluR1 is also a response to chronic activation of glutamate-mediated neurotransmission.

The NR2D subunit is predominantly expressed during development and confers slow channel kinetics to the NMDA receptors (Cull-Candy et al., 2001; Monyer et al., 1994; Vicini and Rumbaugh, 2000). The slow deactivation of the embryonic subunits is believed to lower the temporal threshold for coincidence detection favoring synaptic strengthening during development (Cull-Candy et al., 2001; Monyer et al., 1994; Vicini and Rumbaugh, 2000). Extrasynaptically located NR2D receptors have been demonstrated (Misra et al., 2000). Such extrasynaptic NR2D receptors are thought to mediate glutamate trophic actions rather than contributing to neural transmission (Misra et al., 2000; Vicini and Rumbaugh, 2000). Thus, the increased expression of the embryonic NR2D subunit in the lateral hypothalamus of cocaine escalating rats could be a hallmark of plastic structural rearrangements.

Alterations in the expression of different K+ channels suggest changes in cellular excitability in the LH. Particularly in cocaine-escalating rats, the expressions of a delayed rectifier, an A-type potassium channel (Kv3.4), and an inward rectifier were increased. Delayed rectifiers reduce cellular excitability by increasing action potential threshold, while both delayed rectifiers and A-type channels act by reducing the duration of action potentials resulting in increased frequency of firing (Coetzee et al., 1999). This firing characteristic is usually associated with inhibitory interneurons (Coetzee et al., 1999). The Kv3.4 channel is sparsely expressed, but has been shown to be expressed in the subthalamic nucleus, whose neurons have characteristics of both projection neurons and interneurons and contribute to the regulation of midbrain dopaminergic neurons (Rudy et al., 1999). Inward rectifiers have been involved in opioid inhibition of locus coeruleus neurons (Nestler and Aghajanian, 1997). The Kir4.1 inward rectifier channel has also been implicated in neuronal development and differentiation (Neusch et al., 2001). Increased expression of the vesicular inhibitory amino acid transporter in the LH of cocaine-escalating rats was also observed. The vesicular inhibitory amino acid transporter is a marker of inhibitory synapses (Dumoulin et al., 1999) and its increased expression could suggest increased synaptic terminals from inhibitory interneurons.

The G-protein beta subunit rGbeta1, was found to be downregulated in the LH of escalating rats, Interestingly, this G-protein beta subunit is upregulated by cocaine or amphetamine in the shell region of the nucleus accumbens and it is required for behavioral sensitization induced by repeated administration of psychostimulants (Wang et al., 1997).

The expression levels of only a small fraction of genes changed specifically in association with drug intake escalation (ES genes). The most dramatic changes were observed in the lateral hypothalamus. This observation points to a previously under-appreciated importance of this hypothalamic area in the development of drug addiction. Most of the ES genes identified encode for proteins normally involved in key neurodevelopmental processes, including neurite extension and synaptogenesis differentiation and apoptosis. Genes involved in such processes are increasingly recognized as mediators of plasticity and regeneration in the adult brain. A second broad category of genes that was found to be selectively regulated in cocaine escalating animals are genes involved in the regulation of glutamate neurotransmission and neuronal excitability. The concurrent changes in these two categories of genes during cocaine intake escalation indicates that they are an adaptation to a common perturbation. The present observations show that escalation of cocaine intake is associated with changes in brain structure and function does not depend on a single gene, but on an intricate interplay of multiple genes involved in plastic rearrangement of neural connections and transmission and that neuroadaptative changes in response to chronic activation of glutamate-mediated excitatory neurotransmission could be present in the lateral hypothalamus of rats with escalated cocaine intake.

Behavioral procedure. Twenty-eight male Wistar rats (280-340 g) were prepared with a chronic intravenous catheter and 5 days later were food-restricted and trained for 7 days to press a lever to obtain food pellets. Two days after food-training, 20 rats were tested for cocaine self-administration during two consecutive phases: a screening phase (1 day) and an escalation phase (18 days). The remaining 8 rats were exposed to the same experimental manipulations as the other rats, except that they were not exposed to cocaine. During the screening phase, the 20 rats tested for self-administration were allowed to self-administer cocaine during only one hour on a fixed-ratio 1 schedule (250 μg/injection in a volume of 0.1 ml delivered in 4 sec) after which two balanced groups with the same mean weight and mean cocaine intake were formed. During the escalation phase, one group had access to cocaine self-administration for only 1 hour per day (Short-Access or ShA rats) and the other group for 6 hours per day (Long-Access or LgA rats). Four out of the 20 rats allowed to self-administer cocaine were discarded from the study either because of a failure to reach the criterion for acquisition of cocaine self-administration (n=3) (i.e., at least 8 injections per hour) or because of inconsistent within-session intake for several days (n=1), leaving 8 rats per group.

Brain dissection. Drug-naive, ShA and LgA rats (8 per group) were sacrificed in random order following anesthesia by CO₂ narcosis and perfused with 10% RNA Later (Ambion) in phosphate buffered solution. To reduce variation between animals as much as possible, brains were carefully sliced using a wire brain slicer (Research Instruments & MFG, Corvallis Oreg.). Brain slices were then dissected with the assistance of a brain atlas. Standardized needle punching was performed to remove the nucleus accumbens (ACC), the lateral hypothalamus area (LH), the septum (SEP) and the ventral tegmental area (VTA). The punching needle (14 gauge) was constructed from a modified spinal tap needle and equipped with a plunger. The medial prefrontal cortex (PFC) and the amygdaloid complex (AMG) were dissected free-handedly using established anatomical landmarks. Due to the small size of certain brain regions, tissue samples from different animals had to be pooled. Pools from 2, 4, or 8 animals were made for AMG and MPF, ACC and LH, and SEP and VTA respectively.

RNA and Probe preparation. Total RNA of regions of interest were prepared using the Qiagen RNeasy miniprep kit according to manufacturer's protocol. Quality of RNA was assessed spectrophotometrically and by agarose gel electrophoresis. Between 1 and 5 micrograms of total RNA were used to prepare double-stranded cDNA (1^{st} & 2^{nd} strand cDNA synthesis components from GibcoBRL). Biotinylated cRNA was transcribed from that cDNA using the BioArray High Yield RNA Transcript Labeling kit (Enzo), purified on RNeasy spin columns (Qiagen), and then fragmented.

Hybridization. Hybridization cocktails were boiled at 99° C., loaded on the Affymetrix Neurobiology RNU34 chips, and hybridized at 45° C. for 16 hours. Washes were performed on the Affymetrix Fluidics Station using manufacturer recommended wash solutions and stained with a streptavidin phycoerytin conjugate to allow for fluorescent detection. After staining, chips were scanned with the Affymetrix Chip Reader at 3 μm resolution. For the AMG and PFC, hybridizations were run in quadruplicate (4 independent pools hybridized once each). For the ACC and LH we carried out duplicate hybridizations of 2 pools each (2 independent pools hybridized twice each). For the VTA and SEP, we carried out 3 replicate hybridizations of individual pools (1 pool hybridized 3 times).

Data analysis. Gene expression changes associated with escalated cocaine intake (ES genes) were investigated. ES genes were defined as genes whose expression levels in LgA rats was significantly different (p<0.05) both from control rats and ShA rats. Genes with expression levels different from control levels in both ShA and LgA, but not different between ShA and LgA rats were defined as being associated with cocaine self-administration (SA genes) but not with escalation. Quadriplicate or triplicate results were averaged in each group. Probe sets with mean expression levels below 20 in all three groups were not considered for subsequent analyses and negative expression values were turned to 0. Following previous recommendations (Lockhart and Barlow, 2001), only probe sets displaying significant (p<0.05) changes of 1.8-folds or greater were considered biologically significant. However, probe sets with changes between 1.4 and 1.8 folds were also included if highly significant (p<0.01).

### REFERENCES

Ahmed, S., Walker, J., and Koob, G. (2000) Persistent Increase in the Motivation to Take Heroin in Rats with a History of Drug Escalation. Neuropsychopharmacology 22: 413-421.
Ahmed, S. H., Kenny, P. J., Koob, G. F., and Marko, A. (2002) Neurobiological evidence for hedonic allostasis associated with escalating cocaine use. Nat Neurosci 5: 625-6.
Ahmed, S. H., and Koob, G. F. (1998) Transition from moderate to excessive drug intake: change in hedonic set point. Science 282: 298-300.
Alvaro J D, Tatro J B, Duman R S. Melanocortins and opiate addiction. Life Sci. 1997; 61(1):1-9.
Alvaro J D, Taylor J R, Duman R S. Molecular and behavioral interactions between central melanocorins and cocaine. J Pharmacol Exp Ther. 2003 January; 304(1):391-9.
Bandtlow, C. E., and Zimmermann, D. R. (2000) Proteoglycans in the developing brain: new conceptual insights for old proteins. Physiol Rev 80: 1267-90.
Cagnin A, Gerhard A, Banati R B. The concept of in vivo imaging of neuroinflammation with [11C](R)-PK11195 PET. Ernst Schering Res Found Workshop. 2002; (39):179-91.
Cantallops, I., and Cline, H. T. (2000) Synapse formation: if it looks like a duck and quacks like a duck. Curr Biol 10: R620-3.
Carlezon, W. A., Jr., Boundy, V. A., Haile, C. N., Lane, S. B., Kalb, R. G., Neve, R. L., and Nestler, E. J. (1997) Sensitization to morphine induced by viral-mediated gene transfer. Science 277: 812-4.
Carlezon, W. A., Jr., Todtenkopf, M. S., McPhie, D. L., Pimentel, P., Pliakas, A. M., Stellar, J. R., and Trzcinska, M. (2001) Repeated exposure to rewarding brain stimulation downregulates GluR1 expression in the ventral tegmental area. Neuropsychopharmacology 25: 234-41.
Chapman, G. A., Moores, K., Harrison, D., Campbell, C. A., Stewart, B. R., and Strijbos, P. J. (2000) Fractalkine cleavage from neuronal membranes represents an acute event in the inflammatory response to excitotoxic brain damage. J Neurosci 20: RC87.
Chen J M, Jin G, Sung A, Levin J L Anthranilate sulfonamide hydroxamate TACE inhibitors. Part 1: Structure-based design of novel acetylenic P1′ groups. Bioorg Med Chem Lett. 2002 Apr. 22; 12(8):1195-8.
Coetzee, W. A., Amarillo, Y., Chiu, J., Chow, A., Lau, D., McCormack, T., Moreno, H., Nadal, M. S., Ozaita, A., Pountney, D., Saganich, M., Vega-Saenz de Miera, E., and Rudy, B. (1999) Molecular diversity of K+ channels. Ann NY Acad Sci 868: 233-85.
Conway J G, Andrews R C, Beaudet B, Bickett D M, Boncek V, Brodie T A, Clark R L, Crumrine R C, Leenitzer M A, McDougald D L, Han B, Hedeen K, Lin P, Milla M, Moss M, Pink H, Rabinowitz M H, Tippin T, Scates P W, Selph J, Stimpson S A, Warner J, Becherer J D. Inhibition of tumor necrosis factor-alpha (TNF-alpha) production and arthritis in the rat by GW3333, a dual inhibitor of TNF-alpha-converting enzyme and matrix metalloproteinases. J Pharmacol Exp Ther. 2001 September; 298(3):900-8.
Cull-Candy, S., Brickley, S., and Farrant, M. (2001) NMDA receptor subunits: diversity, development and disease. Curr Opin Neurobiol 11: 327-35.
Duan J J, Chen L, Wasserman Z R, Lu Z, Liu R Q, Covington M B, Qian M, Hardman K D, Magolda R L, Newton R C, Christ D D, Wexler R R, Decicco C P. Discovery of gamma-lactam hydroxamic acids as selective inhibitors of tumor necrosis factor alpha converting enzyme: design, synthesis, and structure-activity relationships. J Med. Chem. 2002 Nov. 7; 45(23):4954-7.
Dumoulin, A., Rostaing, P., Bedet, C., Levi, S., Isambert, M. F., Henry, J. P., Triller, A., and Gasnier, B. (1999) Presence of the vesicular inhibitory amino acid transporter in GABAergic and glycinergic synaptic terminal boutons. J Cell Sci 112: 811-23.
Hooper N M, Karran E H, Turner A J. Membrane protein secretases. Biochem J. 1997 Jan. 15; 321 (Pt 2):265-79.
Hosomi N, Lucero J, Heo J H, Koziol J A, Copeland B R, del Zoppo G J. Rapid differential endogenous plasminogen activator expression after acute middle cerebral artery occlusion. Stroke. 2001 June; 32(6):1341-8.
Huang, M., Page, C., Reynolds, R. K., and Lin, J. (2000) Constitutive activation of stat 3 oncogene product in human ovarian carcinoma cells. Gynecol Oncol 79: 67-73.
Johnson T R, Trojan J, Anthony D D, et al. Gene therapy of rat brain glioblastoma by an episome-based transcriptional cassette expressing antisense IGF-I cDNA. Indian J Biochem Biophys 1994; 31 :1-13.
Kawai, H., Yasuda, H., Terada, M., Omatsu-Kanbe, M., and Kikkawa, R. (1997) Axonal contact regulates expression of alpha2 and beta2 isoforms of Na+, K+-ATPase in Schwann cells: adhesion molecules and nerve regeneration. J Neurochem 69: 330-9.
Koob, G. F., Sanna, P. P., and Bloom, F. E. (1998) Neurobiology of drug addiction. Neuron 21: 467-476.
Letavic M A, Barberia J T, Carty T J, Hardink J R, Liras J, Lopresti-Morrow L L, Mitchell P G, Noe M C, Reeves L M, Snow S L, Stam E J, Sweeney F J, Vaughn M L, Yu C H. Synthesis and biological activity of piperazine-based dual MMP-13 and TNF-alpha converting enzyme inhibitors. Bioorg Med Chem Lett. 2003 Oct. 6; 13(19):3243-6.
Levin J L Chen J M, Cheung K, Cole D, Crago C, Santos E D, Du X, Khafizova G, MacEwan G, Niu C, Salaski E J, Zask A, Cummons T, Sung A, Xu J, Zhang Y, Xu W, Ayral-Kaloustian S, Jin G, Cowling R, Barone D, Mohler K M, Black R A, Skotnicki J S. Acetylenic TACE inhibitors. Part 1. SAR of the acyclic sulfonamide hydroxamates. Bioorg Med Chem Lett. 2003 Aug. 18; 13(16):2799-803.
Levin J I, Chen J M, Du M T, Nelson F C, Killar L M, Skala S, Sung A, Jin G, Cowling R, Barone D, March C J, Mohler K M, Black R A, Skotnicki J S. Anthranilate sulfonamide hydroxamate TACE inhibitors. Part 2: SAR of the acetylenic P1′ group. Bioorg Med Chem Lett. 2002 Apr. 22; 12(8):1199-202.
Levin J I, Chen J M, Du M T, Nelson F C, Wehr T, DiJoseph J F, Killar L M, Skala S, Sung A, Sharr M A, Roth C E, Jin G, Cowling R, Di L, Sherman M, Xu Z B, March C J, Mohler K M, Black R A, Skotnicki J S. The discovery of anthranilic acid-based MMP inhibitors. Part 3: incorporation of basic amines. Bioorg Med Chem Lett. 2001a Nov. 19; 11(22):2975-8.
Levin J I, Chen J, Du M, Hogan M, Kincaid S, Nelson F C, Venkatesan A M, Wehr T, Zask A, DiJoseph J, Killar L M, Skala S, Sung A, Sharr M, Roth C, Jin G, Cowling R, Mohler K M, Black R A, March C J, Skotnicki J S. The discovery of anthranilic acid-based MMP inhibitors. Part 2: SAR of the 5-position and P1(1) groups. Bioorg Med Chem Lett. 2001b Aug. 20; 11(16):2189-92.
Levitzki A. Protein tyrosine kinase inhibitors as novel therapeutic agents. Pharmacol Ther. 1999 May-June; 82(2-3):231-9.
Lindblom J, Wikberg J E, Bergstrom L. Alcohol-preferring AA rats show a derangement in their central melanocortin signalling system Pharmacol Biochem Behav. 2002 May; 72(1-2):491-6.
Lipson K E, Pang L, Huber L J, Chen H, Tsai J M, Hirth P, Gazit A, Levitzli A, McMahon G. Inhibition of platelet-derived growth factor and epidermal growth factor receptor signaling events after treatment of cells with specific synthetic inhibitors of tyrosine kinase phosphorylation. J Pharmacol Exp Ther. 1998 May; 285(2):844-52.
Lockhart, D. J. & Barlow, C. Expressing what's on your mind: DNA arrays and the brain. Nat. Rev. Neurosci. 2, 63-68 (2001).
Lokensgard J R, Hu S, Hegg C C, Thayer S A, Gekker G, Peterson P K Diazepam inhibits HIV-1 Tat-induced migration of human microglia. J Neurovirol. 2001 October; 7(5):481-6.
Lokensgard J R, Chao C C, Gekker G, Hu S, Peterson P K. Benzodiazepines, glia, and HIV-1 neuropathogenesis. Mol Neurobiol. 1998 August; 18(1):23-33.
Misra, C., Brickley, S. G., Farrant, M., and Cull-Candy, S. G. (2000) Identification of subunits contributing to synaptic and extrasynaptic NMDA receptors in Golgi cells of the rat cerebellum. J Physiol 524 Pt 1: 147-62.
Monyer, H., Burnashev, N., Laurie, D. J., Sakmann, B., and Seeburg, P. H. (1994) Developmental and regional expression in the rat brain and functional properties of four NMDA receptors. Neuron 12: 529-40.
Mullberg J, Durie F H, Otten-Evans C, Alderson M R, Rose-John S, Cosman D, Black R A, Mohler K M. A metalloprotease inhibitor blocks shedding of the IL-6 receptor and the p60 TNF receptor. J. Immunol. 1995 Dec. 1; 155(11):5198-205.
Nelson F C, Delos Santos E, Levin J L Chen J M, Skotnicki J S, DiJoseph J F, Sharr M A, Sung A, Killar L M, Cowling R, Jin G, Roth C E, Albright J D. Benzodiazepine inhibitors of the MMPs and TACE. Bioorg Med Chem Lett. 2002 Oct. 21; 12(20):2867-70.
Nestler, E. J., and Aghajanian, G. K (1997) Molecular and cellular basis of addiction. Science 278: 58-63.
Neusch, C., Rozengurt, N., Jacobs, R. E., Lester, H. A., and Kofuji, P. (2001) Kir4.1 potassium channel subunit is crucial for oligodendrocyte development and in vivo myelination. J Neurosci 21: 5429-38.
Nicholas W. Seeds, Mark E. Basham, and Jayne E. Ferguson Absence of Tissue Plasminogen Activator Gene or Activity Impairs Mouse Cerebellar Motor Learning The Journal of Neuroscience, Aug. 13, 2003, 23(19):7368-7375.
Nicole O, Docagne F, Ali C, Margaill L Carneliet P, MacKenzie E T, Vivien D, Buisson A. The proteolytic activity of tissue-plasminogen activator enhances NMDA receptor-mediated signaling. Nat Med. 2001 January; 7(1):59-64.
Parrizas M, Gazit A, Levitzki A, Wertheimer E, LeRoith D. Specific inhibition of insulin-like growth factor-1 and insulin receptor tyrosine kinase activity and biological function by tyrphostins. Endocrinology. 1997 April; 138(4):1427-33.
Ploj K, Roman E, Kask A, Hyytia P, Schioth H B, Wikberg J E, Nylander I. Effects of melanocortin receptor ligands on ethanol intake and opioid peptide levels in alcohol-preferring AA rats. Brain Res Bull. 2002 Oct. 30; 59(2):97-104.
Reizes O, Benoit S C, Strader A D, Clegg D J, Akunuru S, Seeley R J. Syndecan-3 modulates food intake by interacting with the melanocortin/AgRP pathway. Ann N Y Acad. Sci. 2003 June; 994:66-73.
Robbins J R, McGuire P G, Wehrle-Haller B, Rogers S L. Diminished matrix metalloproteinase 2 (MMP-2) in ectomesenchyme-derived tissues of the Patch mutant mouse: regulation of MMP-2 by PDGF and effects on mesenchymal cell migration. Dev Biol. 1999 Aug. 15; 212(2):255-63.
Rudy, B., Chow, A., Lau, D., Amarillo, Y., Ozaita, A., Saganich, M., Moreno, H., Nadal, M. S., Hemandez-Pineda, R., Hernandez-Cruz, A., Erisir, A., Leonard, C., and Vega-Saenz de Miera, E. (1999) Contributions of Kv3 channels to neuronal excitability. Ann N Y Acad Sci 868: 304-43.
Sharon R, Abramovitz R, Miskin R. Plasminogen mRNA induction in the mouse brain after kainate excitation: codistribution with plasminogen activator inhibitor-2 (PAI-2) mRNA. Brain Res Mol Brain Res. 2002 Aug. 15; 104(2):170-5.
Si J, Collins S J. IL-3-induced enhancement of retinoic acid receptor activity is mediated through Stat5, which physically associates with retinoic acid receptors in an IL-3-dependent manner. Blood. 2002 Dec. 15; 100(13):4401-9. Epub 2002 Jun. 28.
Sims, K. D., Straff; D. J., and Robinson, M. B. (2000) Platelet-derived growth factor rapidly increases activity and cell surface expression of the EAAC1 subtype of glutamate transporter through activation of phosphatidylinositol 3-kinase. J Biol Chem 275: 5228-37.
Stamenkovic I. Extracellular matrix remodelling: the role of matrix metalloproteinases. J Pathol. 2003 July; 200(4):448-64. Review.
Stephanou, X, Brar, B. K., Knight, R A., and Latchman, D. S. (2000) Opposing actions of STAT-1 and STAT-3 on the Bcl-2 and Bcl-x promoters. Cell Death Differ 7: 329-30.
Toyonaga T, Nakano K, Nagano M, Zhao G, Yamaguchi K, Kuroki S, Eguchi T, Chijiiwa K, Tsuneyoshi M, Tanaka M. Blockade of constitutively activated Janus kinase/signal transducer and activator of transcription-3 pathway inhibits growth of human pancreatic cancer. Cancer Lett. 2003 Nov. 10; 201(1):107-16.
Versijpt J J, Dumont F, Van Laere K J, Decoo D, Santens P, Audenaert K, Achten E, Slegers G, Dierckx R A, Korf J. Assessment of neuroinflammation and microglial activation in Alzheimer's disease with radiolabelled PK11195 and single photon emission computed tomography. A pilot study. Eur Neurol. 2003; 50(1):39-47.
Vicini, S., and Rumbaugh, G. (2000) A slow NMDA channel: in search of a role. J Physiol 525 Pt 2: 283.
Wang, X. B., Funada, M., Imai, Y., Revay, R. S., Ujike, H., Vandenbergh, D. J., and Uhl, G. R. (1997) rGbeta1: a psychostimulant-regulated gene essential for establishing cocaine sensitization. J Neurosci 17: 5993-6000.
Zask A, Gu Y, Albright J D, Du X, Hogan M, Levin J I, Chen J M, Killar L M, Sung A, DiJoseph J F, Sharr M A, Roth C E, Skala S, Jin G, Cowling R, Mohler K M, Barone D, Black R, March C, Skotnicki J S. Synthesis and SAR of bicyclic heteroaryl hydroxamic acid MMP and TACE inhibitors. Bioorg Med Chem Lett. 2003 Apr. 17; 13(8):1487-90.
Zhang Y, Turkson J, Carter-Su C, Smithgall T, Levitzki A, Kraker A, Krolewski J J, Medveczky P, Jove R. Activation of Stat3 in v-Src-transformed fibroblasts requires cooperation of Jak1 kinase activity. J Biol. Chem. 2000 Aug. 11; 275(32):24935-44.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

TABLE 1
RESULTS: Micro Array Data Analysis

| | | | | | | | | | | Scheffe's | | | | | |
|---|---|---|---|---|---|---|---|---|---|---|---|---|---|---|---|
| | | | | | | | Fisher's post-hoc | | | post-hoc | | | | | |
| | UniGene | | Brain | Di- | FC | | Ctrl | Ctrl | BhA | Ctrl | | ShA | Flod Changes | | |
| Gene | Annotation | Accession | Region | rection | Level | Gene Type | vz ShA | vs LoA | vs LoA | vs ShA | Ctrl vs LA | vs LA | [symbol] /Ctrl | [symbol] /Ctrl | [symbol] |

Growth/Structure/Plasticity

| | | | | | | | | | | | | | | | |
|---|---|---|---|---|---|---|---|---|---|---|---|---|---|---|---|
| Tumor [symbol] crosis factor | | M63122 | ACC | Down | −−− | Growth/Structure/ | p < 0.05 | p < 0.01 | p < 0.002 | NS | p < 0.01 | NS | 0.00 | 0.38 | 0.66 |
| receptor chain (p60) | | | | | | Plasticity | | | | | | | | | |
| [symbol] -like growth factor II | 1gt2 insulin-like | X17912 | AMG | Down | −−− | Growth/Structure/ | p < 0.01 | p < 0.01 | p < 0.05 | p < 0.05 | p < 0.01 | NS | 0.61 | 0.36 | 0.59 |
| | growth factor if | | | | | Plasticity | | | | | | | | | |
| | ( [symbol] A) | | | | | | | | | | | | | | |
| [symbol] | Rattus norvegicus | AP038352/ | LH | Up | ++ | Growth/Structure/ | p < 0.01 | p < 0.01 | p < 0.01 | p < 0.01 | p < 0.01 | p < 0.01 | 1.40 | 1.91 | 1.26 |
| CX3C [symbol] ligand 1 | [symbol] CX3C nRNA, | A1227547 | | | | Plasticity | | | | | | | | | |
| | [symbol] (molecule) | | | | | | | | | | | | | | |
| | secreted [symbol] /cDNA | | | | | | | | | | | | | | |
| | clone R [symbol] 70 [symbol] | | | | | | | | | | | | | | |
| | [symbol] (C-X3-C [symbol] | | | | | | | | | | | | | | |
| | ligand 1; SYD1_RAT | | | | | | | | | | | | | | |
| | [symbol] | | | | | | | | | | | | | | |
| | (CX3Ct.1) ( [symbol] ) | | | | | | | | | | | | | | |
| | (CX3C [symbol] -enchored | | | | | | | | | | | | | | |
| | ct [symbol] kine) (S$4 t [symbol] | | | | | | | | | | | | | | |
| | molecule) | | | | | | | | | | | | | | |
| Pla [symbol] -derived growth | Pdgfa Plast [symbol] -derived | D10106 | LH | Up | + | Growth/Structure/ | p < 0.01 | p < 0.01 | p < 0.01 | p < 0.01 | p < 0.01 | p < 0.05 | 1.37 | 1.39 | 1.36 |
| factor A ctchani | growth factor A chain | | | | | Plasticity | | | | | | | | | |
| (PDGFa) | | | | | | | | | | | | | | | |
| Bcl- [symbol] alpha (Bcl-2 | | U72350 | LH | Up | +++ | Growth/Structure/ | NS | p < 0.01 | p < 0.01 | NS | p < 0.01 | p < 0.01 | 1.25 | 2.13 | 1.7 |
| fa [symbol] y) | | | | | | Plasticity | | | | | | | | | |
| Signal transducer and | | X91310 | LH | Up | +++ | Growth/Structure/ | p < 0.078 | p < 0.01 | p < 0.01 | NS | p < 0.01 | p < 0.01 | 1.76 | 3.4 | 1.34 |
| activator of | | | | | | Plasticity | | | | | | | | | |
| transcription | | | | | | | | | | | | | | | |
| 3 (STAT 3) | | | | | | | | | | | | | | | |
| N-sydacase | Sdcs Syndecase | X [symbol] 143 | LH | Up | +++ | Growth/Structure/ | p < 0.01 | p < 0.01 | p < 0.01 | p < 0.01 | p < 0.01 | p < 0.01 | 4.94 | 8.61 | 1.74 |
| (Neuroglycan) | | | | | | Plasticity | | | | | | | | | |
| Microtu [symbol] -associated | | M [symbol] 3194 | LH | Up | +++ | Growth/Structure | p < 0.81 | p < 0.91 | p < 0.91 | p < 0.01 | p < 0.01 | p < 0.01 | 1.63 | 2.19 | 1.33 |
| prot [symbol] (MAP1A) | | | | | | Plasticity | | | | | | | | | |
| Ra [symbol] - [symbol] QTPase (R [symbol] 3) | | X [symbol] | LH | Up | ++ | Growth/Structure/ | p < 0.01 | p < 0.01 | p < 0.01 | p < 0.01 | p < 0.01 | p < 0.01 | 1.35 | 2.82 | 1.5 |
| | | | | | | Plasticity | | | | | | | | | |
| [symbol] -like growth factor II | fgt2 la [symbol] -like growth | E [symbol] | SEP | Down | + to − | Growth/Structure | NS | p < 0.01 | p < 0.06 | NS | p < 0.05 | p < 0.05 | 0.72 | −0.42 | −0.58 |
| | factor if (somef [symbol] A) | | | | | Plasticity | | | | | | | | | |
| Microtubus-aasociated | Map1b Microtubu [symbol] - | U52850 | SEP | Down | −−− | Growth/Structure/ | NS | p < 0.01 | p < 0.05 | NS | p < 0.05 | p < 0.05 | 0.89 | 0.42 | 0.54 |
| proteins (MAP1B) | associated protein 1b | | | | | Plasticity | | | | | | | | | |
| Metrollgin 3 | | U41682 | SEP | Down | | Growth/Structure/ | p < 0.72 | p < 0.05 | NS | NS | p < 0.62 | p < 0.06 | Need | | |
| | | | | | | Plasticity | | | | | | | FC | | |
| [symbol] leukin-3 beta | | U81482 | SEP | Up | ++ | Growth/Structure/ | NS | p < 0.05 | p < 0.05 | NS | p < 0.05 | p < 0.05 | 1.01 | 1.34 | 1.83 |
| | | | | | | Plasticity | | | | | | | | | |
| Rstlno [symbol] na protein | | [symbol] 26283 | SEP | Up | + | Growth/Structure/ | p < 0.05 | p < 0.01 | p < 0.01 | p < 0.06 | p < 0.01 | p < 0.89 | 1.2 | 1.39 | 1.35 |
| (pftb) | | | | | | Plasticity | | | | | | | | | |
| beta-S-100 Ca++ binding | | AM46214 | VTA | Up | +++ | Growth/Structure/ | p < 0.05 | p < 0.01 | p < 0.01 | p < 0.76 | p < 0.01 | p < 0.01 | 1.36 | 2.39 | 1.78 |
| protein | | | | | | Plasticity | | | | | | | | | |

Ion pumps and channels

| | | | | | | | | | | | | | | | |
|---|---|---|---|---|---|---|---|---|---|---|---|---|---|---|---|
| K+ c [symbol] beta subunit | | X7 [symbol] 2 | ACC | Down | ++ | Ion pumps and | NS | p < 0.01 | p < 0.05 | NS | p < 0.01 | p < 0.004 | 0.5 | 0.61 | 0.63 |
| ( [symbol] Ctrl-type) | | | | | | channels | | | | | | | | | |
| Na+ channel beta 2 | Scn2b Sodium channel | U37147 | AMG | Up | +++ | Ion pumps and | NS | p < 0.05 | p < 0.05 | NS | p < 0.05 | NS | 1.25 | 1 | 1.61 |
| subunit (Sort2b) | beta 2 | | | | | channels | | | | | | | | | |
| Na+, K+-transporting | Atp1a2 ATPase, | M22849 | LH | Up | +++ | Ion pumps and | p < 0.01 | p < 0.01 | p < 0.05 | p < 0.01 | p < 0.01 | p < 0.94 | 2.45 | 3.12 | 1.27 |
| ATPase alpha 2 subunit | Na+K+ transporting, | | | | | channels | | | | | | | | | |
| (Atp1 [symbol] 2) | alpha 2 | | | | | | | | | | | | | | |
| Voltage g [symbol] d K+ C [symbol] | K [symbol] P [symbol] Voltage | X82941 | LH | Up | ++ | Ka pumps and | NS | p < 0 0.01 | p < 0.01 | NS | p < 0.01 | p < 0.01 | 1.36 | 1.82 | 1. [symbol] 9 |
| K [symbol] 3,4 [symbol] | [symbol] | | | | | channats | | | | | | | | | |
| [symbol] | [symbol] | | | | | | | | | | | | | | |
| K+ [symbol] | | XZ762 [symbol] | LH | Up | +++ | Ka pumps and | NS | p < 0.01 | p < 0.01 | NS | p < 0.01 | p < 0.05 | 2.34 | 2.34 | 1.96 |
| [symbol] | | X82689 | | | | channats | | | | | | | | | |
| [symbol] a ATP [symbol] beta [symbol] | | [symbol] | LH | Up | +++ | Ka pumps and | p < 0. [symbol] | p < 0.01 | p < 0.81 | NS | p < 0.01 | p < 0.01 | 1.41 | 2.36 | 2. [symbol] |
| [symbol] | | | | | | channats | | | | | | | | | |
| K+ [symbol] 10 | | X [symbol] | LH | Up | + | Ka pumps and | p < 0.01 | p < 0.01 | p < 0.01 | p < 0.96 | p < 0.01 | p < 0.01 | 0.74 | 1.3 | 2.75 |
| (K [symbol] ) | | | | | | channats | | | | | | | | | |
| Ca++ [symbol] alpha 1 | [symbol] | U14 [symbol] 06 | VTA | Up | +++ | K [symbol] pumps and | NS | p < 0.01 | p < 0.01 | NS | p < 0.01 | p < 0.01 | [symbol] | [symbol] | [symbol] |
| subunit (Channets) | | alpha 1A | | | | | channats | | | | | | | | |

Neurotransmitters/Receptors/Enzymes/Transporters

| | | | | | | | | | | | | | | | |
|---|---|---|---|---|---|---|---|---|---|---|---|---|---|---|---|
| EST [symbol] | Ul-a-Cl- [symbol] | A [symbol] 064517 | ACC | Down | −−− | Naurotramitters/ | NS | p < 0.0 [symbol] | p < 0.05 | NS | p < 0.074 | p < 0.05 | 1.06 | 0.39 | 0.37 |
| [symbol] | Cl [symbol] | | | | | Receptors/Enzymes/ | | | | | | | | | |
| [symbol] transporters (GLT | [symbol] | | | | | Transporters | | | | | | | | | |
| 1A) | MRNA | | | | | | | | | | | | | | |
| | [symbol] | | | | | | | | | | | | | | |
| | [symbol] | | | | | | | | | | | | | | |
| | [symbol] | | | | | | | | | | | | | | |
| | [symbol] | | | | | | | | | | | | | | |
| | [symbol] | | | | | | | | | | | | | | |
| Catach [symbol] | | M60763 | A [symbol] | Down | −−− | Neurotransmitters/ | NS | p < 0.05 | p < 0.005 | NS | p < 0.083 | p < 0.074 | 0. [symbol] | 0.45 | 0.52 |
| [symbol] | | | | | | Receptors/Enzymes/ | | | | | | | | | |
| | | | | | | Transporters | | | | | | | | | |
| A [symbol] receptor [symbol] | [symbol] receptor | X17184 | LH | Down | −−− | Neurotransmitters/ | NS | p < 0.01 | p < 0.01 | NS | p < 0.05 | p < 0.01 | 1.11 | 0.52 | 0.46 |
| | [symbol] AMPA1 (alpha 1); | | | | | Receptors/Enzymes/ | | | | | | | | | |
| | X17 [symbol] 4 [symbol] | | | | | Transporters | | | | | | | | | |
| | [symbol] MRNA for | | | | | | | | | | | | | | |
| | [symbol] receptor, AMPA | | | | | | | | | | | | | | |
| | subtype [symbol] | | | | | | | | | | | | | | |
| K [symbol] receptor subunit | [symbol] receptor, | X [symbol] | LH | Down | −−− | Neurotransmitters/ | NS | p < 0.01 | p < 0.01 | NS | p < 0.01 | p < 0.05 | 0.34 | 0.26 | 0.33 |
| (KA1) | [symbol] 4 | | | | | Receptors/Enzymes/ | | | | | | | | | |
| | | | | | | Transporters | | | | | | | | | |
| G-protein beta-1 subunit | [symbol] | A227000 | LH | Down | −−− | Neurotransmitters/ | p < 0.01 | p < 0.01 | p < 0.01 | p < 0.01 | p < 0.01 | p < 0.05 | 0.61 | 0.43 | 0. [symbol] |
| [symbol] 1 | [symbol] binding protein bets | | | | | Receptors/Enzymes/ | | | | | | | | | |
| | 1(Gnb [symbol] ) | | | | | Transporters | | | | | | | | | |
| KMDA receptor subunit | | U [symbol] 2 [symbol] | LH | Up | ++ | Neurotransmitters/ | p < 0.01 | p < 0.01 | p < 0.05 | p < 0.01 | p < 0.01 | p < 0. [symbol] | 2. [symbol] 3 | 2.56 | 2.36 |
| 2D [symbol] | | | | | | Receptors/Enzymes/ | | | | | | | | | |
| | | | | | | Transporters | | | | | | | | | |
| [symbol] | | J0 [symbol] 122 | LH | Up | +++ | Neurotransmitters/ | NS | p < 0.05 | p < 0.31 | NS | p < 0. [symbol] | NS | 1.32 | [symbol] .77 | [symbol] |
| [symbol] | | | | | | Receptors/Enzymes/ | | | | | | | | | |
| | | | | | | Transporters | | | | | | | | | |
| beta- [symbol] receptor | [symbol] receptor | [symbol] 4 [symbol] | LH | Up | +++ | Neurotransmitters/ | p < 0.05 | p < 0.01 | p < 0.05 | p < 0.05 | p < 0.01 | p < 0.070 | 1.47 | 2.32 | 1.34 |
| [symbol] | Kinase beta 1 | | | | | Receptors/Enzymes | | | | | | | | | |
| | | | | | | Transporters | | | | | | | | | |
| [symbol] | Pcp4 [symbol] | K24 [symbol] 52 | LH | Down | −− | Neurotransmitters/ | p < 0.01 | p < 0.01 | p < 0.05 | p < 0.01 | p < 0.01 | p < 0.05 | 0.64 | 0.51 | 0.79 |
| (PEP-19) | PEP-19( [symbol] cell protain | | | | | Receptors/Enzymes/ | | | | | | | | | |
| | | | | | | Transporters | | | | | | | | | |
| [symbol] | DdoDopa | [symbol] | LH | Down | −−− | Neurotransmitters/ | NS | p < 0.05 | p < 0.0 [symbol] | NS | NS | p < 0.05 | 0.96 | [symbol] | 0.1 |
| Dopa) decarboxytase | dacarboxytase (aro [symbol] | | | | | Receptors/Enzymes | | | | | | | | | |
| | L-amino acid | | | | | Transporters | | | | | | | | | |
| | decarboxytase); | | | | | | | | | | | | | | |
| | [symbol] MRMA | | | | | | | | | | | | | | |
| | RATAADC01 | | | | | | | | | | | | | | |
| | [symbol] | | | | | | | | | | | | | | |
| | [symbol] L-amino | | | | | | | | | | | | | | |
| | acid decarboxytase | | | | | | | | | | | | | | |
| | gene ax [symbol] ta | | | | | | | | | | | | | | |
| MMDA receptors | | S [symbol] 73 | LH | Up | ++ | Neurotransmitters/ | p < 0.01 | p < 0.01 | p < 0.01 | p < 0.01 | p < 0.01 | p < 0.01 | 1.41 | 1.94 | 1.3 |
| subunit [symbol] Complex | | | | | | Receptors/Enzymes | | | | | | | | | |
| [symbol] -binding | | | | | | Transporters | | | | | | | | | |
| protein(G [symbol] ) | | | | | | | | | | | | | | | |
| alpha [symbol] receptors | | [symbol] 2372 | LH | Up | | Neurotransmitters/ | NS | p < 0.01 | p < 0.01 | NS | p < 0.01 | p < 0.01 | 1.3 [symbol] | 2. [symbol] 179 | 2.0 [symbol] |
| (RG20) | | | | | | Receptors/Enzymes | | | | | | | | | |
| | | | | | | Transporters | | | | | | | | | |
| Vesl [symbol] amino | | AA [symbol] 51 | LH | Up | +++ | Neurotransmitters/ | NS | p < 0.01 | p < 0.01 | NS | p < 0.01 | p < 0.0 [symbol] | 1.33 | [symbol] | 1.6 |
| acid transporters | | | | | | Receprots/Enzymes | | | | | | | | | |
| (5VAAT) | | | | | | Transporters | | | | | | | | | |
| GABAA receptor alpha | | XS1981 | SEP | Down | −−− | [symbol] | NS | p < 0.01 | p < 0.01 | NS | p < 0.05 | p < [symbol] | 0.3719187 | 0.45 | 0.47 |
| 3 subunit ( [symbol] ) | | | | | | [symbol] | | | | | | | | | |
| | | | | | | Transporters | | | | | | | | | |

Release Machinery

| | | | | | | | | | | | | | | | |
|---|---|---|---|---|---|---|---|---|---|---|---|---|---|---|---|
| EST139376 | ESTs, Moderately | AA79879 | AMG | Up | − to + | Release Machinery | NS | p < 0.06 | p < 0.05 | NS | p = 0.083 | p = 0.074 | 2.6 | −7.7 | −2.36 |
| | similar to SNG1_RAT | | | | | | | | | | | | | | |
| | SYNAPTOGYRIN | | | | | | | | | | | | | | |
| | 1(P23) | | | | | | | | | | | | | | |
| [symbol] | D24512 RAT [symbol] Rat | D26812 | LH | Up | +++ | Release Machinery | NS | p < 3.81 | p < 0.01 | NS | p < 0.81 | p < 0.85 | 1.13 | 2.83 | 1.6 |
| | mRNA for [symbol] , | | | | | | | | | | | | | | |
| | complete cds | | | | | | | | | | | | | | |
| Synapto [symbol] XI | membrane traffolding | AF000423 | LH | Up | ++ | Release Machinery | p < 0.045 | p < 0.01 | p < 0.81 | NS | p < 0.01 | p < 0.01 | 0.87 | 1.39 | 2.28 |
| | protects [symbol] Intra [symbol] | | | | | | | | | | | | | | |
| | membrane protein with | | | | | | | | | | | | | | |
| | single transmembrane | | | | | | | | | | | | | | |
| | region and two | | | | | | | | | | | | | | |
| | C3-domains | | | | | | | | | | | | | | |
| ESTs, [symbol] -processed | | AA [symbol] 149 | LH | Down | −−− | Release Machinery | p < 0.01 | p < 0.81 | p < 8.86 | p < 0.91 | p < 0.01 | NS | 0.56 | 0.45 | 0.31 |
| [symbol] Hbeta mRNA | | | | | | | | | | | | | | | |
| Synapto [symbol] VIII | Syts Synapto [symbol] | U20110 | VTA | Up | +++ | Release Machinery | NS | p < 3.01 | p < 3.91 | NS | p < 0.01 | p < 0.81 | 1.3 | 3.82 | 2.71 |

[image] [image] [image] [image] [image] [image] [image] [image] [image] [image] [image] [image] [image] [image] [image] [image] TABLE 3
Lateral Hypothalamus_230Achip

| [symbol] | [symbol] | [symbol] | [symbol] | [symbol] | [symbol] |
|---|---|---|---|---|---|
| 1367799_at | NM_012660 | 2.06 | 1.10 | Statin-like protein | MAS |
| 1367835_at | NM_019279 | 1.90 | 1.04 | proprotein convertase subtilisin/kexin type 1 Inhibitor | MAS |
| 1367868_at | NM_031708 | 3.79 | 1.13 | adhesion regulating molecule 1 | MAS |
| 1367959_a_{—} | AF182949 | 2.98 | 1.14 | sodium channel, voltage-gated, type 1, beta polypeptide | MAS |
| 1368057_at | NM_012804 | −1.52 | −1.07 | ATP-binding cassette, sub-family D (ALD), member 3 | MAS |
| 1368082_at | NM_017048 | 1.94 | 1.18 | Solute carrier family 4, member 2, anion exchange protein 2 | MAS |
| 1368359_a_{—} | NM_030997 | 3.31 | 1.02 | VGF nerve growth factor inducible | MAS |
| 1368417_at | NM_019350 | 1.47 | 1.40 | synaptotagmin 5 | MAS |
| 1368425_at | NM_080690 | 1.64 | 1.08 | cask-Interacting protein 1 | MAS |
| 1368444_at | NM_022703 | 1.42 | 1.38 | small glutamin-rich tetratricopeptide repeat (TPR) containing protein | MAS |
| 1368862_at | NM_033230 | 1.91 | 1.17 | v-akt murine thymoma viral oncogene homolog 1 | MAS |
| 1368951_at | NM_022797 | 5.67 | 1.88 | glutamate receptor, ionotropic, NMDA2D | MAS |
| 1368959_at | NM_017294 | 20.72 | 6.42 | protein kinase C and casein kinase substrate in neurons 1 | MAS |
| 1369128_at | NM_017262 | 1.88 | 1.06 | Glutamate receptor, Ionotropic, kainate 5 | MAS |
| 1369453_at | NM_057136 | 4.36 | 1.35 | Epsin 1 | MAS |
| 1369772_at | AW141210 | 1.71 | 1.34 | glycine transporter 1 | MAS |
| 1369816_at | NM_013018 | 1.96 | 1.54 | Ras-related small GTP binding protein 3A | MAS |
| 1369926_at | NM_022525 | 1.60 | −1.05 | plasma glutathione peroxidase precursor | MAS |
| 1369974_at | NM_012663 | 2.51 | 1.62 | vesicle-associated membrane protein 2 | MAS |
| 1369999_a_{—} | NM_053601 | 1.58 | 1.11 | neuronatin | MAS |
| 1370341_at | AF019973 | 1.65 | 1.21 | enolase 2, gamma | MAS |
| 1370427_at | L06238 | 2.17 | 1.23 | Platelet-derived growth factor A chain | MAS |
| 1370519_at | U06069 | 2.19 | 1.58 | Syntaxin binding protein 1 | MAS |
| 1370922_at | L15011 | 2.47 | −1.08 | cortexin | MAS |
| 1370938_at | AI535144 | 1.90 | 1.18 | Rattus norvegicus reg I binding protein I (Rbp1) mRNA, partial cds | MAS |
| 1370964_at | BF283458 | 1.83 | −1.01 | arginosuccinate synthetase 1 | MAS |
| 1371063_at | AF009603 | 1.62 | 1.25 | SH3 domain protein 2A | MAS |
| 1371104_at | AF286470 | 1.82 | 1.19 | — | MAS |
| 1371359_at | BG381670 | 1.71 | 1.12 | ESTs, Highly similar to MLF2_MOUSE Myeloid leukemia factor 2 (My [symbol] | MAS |
| 1371528_at | BI274519 | 2.07 | 1.11 | ESTs, Highly similar to FKB8_MOUSE 38 kDa FK-506 binding protein | MAS |
| 1371578_at | AW915101 | 1.98 | 1.44 | ESTs | MAS |
| 1371716_at | BE107610 | 1.40 | 1.07 | ESTs | MAS |
| 1372703_at | BG380680 | 1.47 | 1.09 | ESTs, Weakly similar to ubiquitin conjugating enzyme [Rattus norveg [symbol] | MAS |
| 1373470_at | BM388896 | −1.44 | −1.03 | ESTs | MAS |
| 1373787_at | AA943735 | 1.77 | −1.02 | glycine transporter 1 | MAS |
| 1375149_at | AI145991 | 2.77 | 1.08 | ESTs, Highly similar to T46266 hypothetical protein DKFZp761A179,1 | MAS |
| 1375307_at | BI275772 | 1.72 | 1.06 | ESTs, Highly similar to RIKEN cDNA 1200013A08 [Mus musculus] [ [symbol] | MAS |
| 1375612_at | AA965147 | −1.97 | −1.00 | heterogeneous nuclear ribonucleoprotein A1 | MAS |
| 1375657_at | BE107438 | 2.74 | −1.10 | ESTs | MAS |
| 1375720_at | AI171785 | 1.65 | 1.03 | ESTs, Highly similar to GBR1_RAT Gamma-aminobutyric acid type B | MAS |
| 1376233_at | AI144891 | 1.66 | 1.11 | ESTs | MAS |
| 1376345_at | BG381734 | 1.54 | 1.04 | calcyon; D1 dopamine receptor-interacting protein | MAS |
| 1376904_at | AI718115 | 2.78 | 1.08 | ESTs | MAS |
| 1382915_at | AI237079 | −4.24 | −1.02 | ESTs | MAS |
| 1383161_a_{—} | AI008646 | −1.63 | −1.29 | — | MAS |
| 1386874_at | NM_017161 | −1.40 | −1.09 | ribosomal protein S15 | MAS |
| 1386892_at | NM_031975 | 2.57 | 1.30 | parathymosin | MAS |
| 1386909_a_{—} | AF268467 | 1.65 | 1.70 | voltage-dependent anion channel 1 | MAS |
| 1386955_at | BM387903 | 2.20 | 1.46 | glycoprotein lb (platelet), beta polypeptide | MAS |
| 1387429_at | NM_012776 | 2.06 | 1.20 | adrenergic receptor kinase, beta 1 | MAS |
| 1388030_a_{—} | AF312319 | 2.64 | 1.66 | gamma-aminobutyric acid (GABA) B receptor, 1 | MAS |
| 1388088_a_{—} | AB035650 | 7.42 | 1.18 | transcription factor USF2 | MAS |
| 1388158_at | BG057565 | 1.50 | −1.02 | HLA-B-associated transcript 1A | MAS |
| 1388309_at | BG378885 | 1.92 | 1.22 | ESTs | MAS |
| 1388430_at | BI280292 | 1.54 | 1.02 | ESTs, Highly similar to prostate tumor over expressed gene 1 [Homo [symbol] | MAS |
| 1389059_at | BI278651 | 2.63 | −1.29 | ESTs | MAS |
| 1389240_at | AW527026 | 1.92 | 1.21 | ESTs | MAS |
| 1389301_at | AI176665 | −1.47 | −1.09 | ESTs | MAS |
| 1390033_at | BG378062 | 1.78 | 1.21 | ESTs | MAS |
| 1390167_at | BI286834 | 2.58 | −1.07 | ESTs | MAS |
| 1390262_a_{—} | AI705744 | 6.01 | 1.37 | ESTs, Weakly similar to nuclear GTPase PIKE [Rattus norveglcus] [R [symbol] | MAS |
| 1391676_at | AI511097 | −2.53 | 1.03 | ESTs | MAS |
| 1387823_at | BF523128 | 1.90 | 1.35 | tissue inhibitor of metalloproteinase 2 | MAS |
| 1389836_a_{—} | AI599265 | 1.38 | 1.09 | Tissue inhibitor of metalloproteinase 3 | dChip |
| 1367800_at | NM_013151 | 1.28 | 1.20 | Plasminogen activator, tissue | dChip |
| 1373672_at | BM384419 | −1.22 | −1.12 | ESTs, Weakly similar to plasminogen activator Inhibitor 2 type A [Rati [symbol] | dChip |

TABLE 7

| [symbol] | [symbol] | [symbol] | [symbol] | [symbol] | [symbol] |
|---|---|---|---|---|---|
| SP | AA848563_s_at | AA848563 | 1.943548 | −1.116183 | heat shock 70 kD protein 1A |
| ACC | AA851136_g_at | AA851136 | −1.236515 | −1.467442 | p21 (CDKN1A)-activated kinase 1 |
| SP | AA944099_s_at | AA944099 | −1.549947 | 1.259603 | platelet derived growth factor receptor, alpha polypeptide |
| SP | AB002801_at | AB002801 | 8.777778 | 2.089947 | cyclic nucleotide gated channel alpha 3 |
| VTA | AB004638_at | AB004638 | 1.275242 | 2.254279 | fibroblast growth factor 18 |
| VTA | AB013130_at | AB013130 | 1.379007 | 1.640221 | synaptopodin |
| SP | AB013890_at | AB013890 | −2.09417 | −1.982063 | potassium inwardly-rectifying channel, subfamily J, member 13 |
| VTA | AB016161cds_i_at | AB016161 | −1.147732 | 1.032839 | gamma-aminobutyric acid (GABA) B receptor, 1 |
| SP | AF000368_at | AF000368 | 1.294253 | −1.095915 | sodium channel, voltage-gated, type 9, alpha polypeptide |
| VTA | AF000368_at | AF000368 | 1.153766 | 1.510959 | sodium channel, voltage-gated, type 9, alpha polypeptide |
| AMY | AF003598_at | AF003598 | 1.364816 | 1.426526 | integrin beta 7 |
| ACC | AF003825_s_at | AF003825 | 1.418502 | −1.096273 | glial cell line derived neurotrophic factor family receptor alpha 2 |
| AMY | AF007758_at | AF007758 | 1.419998 | 1.041801 | synuclein, alpha |
| SP | AF012347_at | AF012347 | 1.989991 | 1.219064 | MAD homolog 9 (Drosophila) |
| SP | AF014365_s_at | AF014365 | 3.825 | 1.085106 | CD44 antigen |
| AMY | AF015728_s_at | AF015728 | 1.240782 | 1.640325 | cyclic nucleotide-gated channel beta subunit 1 |
| AMY | AF017637_at | AF017637 | −4.855505 | −2.025229 | carboxypeptidase Z |
| VTA | AF021137_s_at | AF021137 | −3.075949 | 1.490566 | potassium inwardly-rectifying channel, subfamily J, member 2 |
| AMY | AF021935_at | AF021935 | 1.029123 | −1.095663 | Ser-Thr protein kinase related to the myotonic dystrophy protein kinase |
| MPF | AF022083_s_at | AF022083 | −1.049755 | 1.223144 | guanine nucleotide binding protein, beta 1 |
| VTA | AF022083_s_at | AF022083 | −1.074567 | 1.837131 | guanine nucleotide binding protein, beta 1 |
| SP | AF025670_g_at | AF025670 | 1.020737 | 1.701665 | caspase 6 |
| VTA | AF027506_s_at | AF027506 | −1.709641 | −1.470852 | solute carrier family 24 (sodium/potassium/calcium exchanger), member 2 |
| SP | AF028603_s_at | AF028603 | 1.02444 | 2.453659 | purinergic receptor P2X, ligand-gated ion channel, 2 |
| AMY | AF030086UTR#1_at | AF030086 | −2.072289 | 2.371429 | Rattus norvegicus activity and neurotransmitter-induced early gene 1 |
| | | | | | (ania-1) mRNA, 3′UTR |
| AMY | AF034896_f_at | AF034896 | −1.413559 | −1.077966 | olfactory receptor-like protein |
| AMY | AF039218_at | AF039218 | −1.110863 | 1.248111 | citron |
| AMY | AF041246_at | AF041246 | 1.778409 | −1.231629 | hypocretin receptor 2 |
| VTA | AF042499_at | AF042499 | 2.070876 | 3.175889 | MAD homolog 7 (Drosophila) |
| ACC | AF049239_s_at | AF049239 | −1.121527 | −1.097757 | sodium channel, voltage-gated, type 8, alpha polypeptide |
| SP | AF049239_s_at | AF049239 | 1.011791 | 1.056272 | sodium channel, voltage-gated, type 8, alpha polypeptide |
| ACC | AF050659UTR#1_at | AF050659 | 1.251876 | 1.093633 | Rattus norvegicus activity and neurotransmitter-induced early gene 7 (ania-7) |
| | | | | | mRNA, 3′ UTR |
| MPF | AF050659UTR#1_at | AF050659 | 2.673913 | 1.356618 | Rattus norvegicus activity and neurotransmitter-induced early gene 7 (ania-7) |
| | | | | | mRNA, 3′ UTR |
| SP | AF050663UTR#1_at | AF050663 | 1.058716 | −1.115425 | Rattus norvegicus activity and neurotransmitter-induced early gene 11 |
| | | | | | (ania-11) mRNA, 3′ UTR |
| VTA | AF050664UTR#1_at | AF050664 | −1.181034 | −1.646552 | Rattus norvegicus activity and neurotransmitter-induced early gene 12 |
| | | | | | (ania-12) mRNA, 3′ UTR |
| VTA | AF052540_s_at | AF052540 | −2.174825 | −2.664336 | calpain 3 |
| SP | AF056704_at | AF056704 | 1.898601 | 1.916471 | synapsin 3 |
| ACC | AF060173_at | AF060173 | −1.286291 | −1.077396 | SV2 related protein |
| SP | AF065432_s_at | AF065432 | 2.702422 | 1.528376 | BCL2-like 11 (apoptosis facilitator) |
| VTA | AF065432_s_at | AF065432 | 1.624473 | −1.94026 | BCL2-like 11 (apoptosis facilitator) |
| ACC | AF065433_at | AF065433 | −1.065119 | −1.263503 | BCL2-like 11 (apoptosis facilitator) |
| MPF | AF065433_at | AF065433 | −1.306889 | −1.519833 | BCL2-like 11 (apoptosis facilitator) |
| SP | AF065433_at | AF065433 | 1.53501 | 1.502461 | BCL2-like 11 (apoptosis facilitator) |
| VTA | AF074482_s_at | AF074482 | 1.201839 | 1.248329 | G protein-coupled receptor 51 |
| AMY | AF075382_at | AF075382 | −1.246106 | −1.990654 | cytokine inducible SH2-containing protein 2 |
| MPF | AF075382_at | AF075382 | 1.899471 | 1.365019 | cytokine inducible SH2-containing protein 2 |
| MPF | AF081366_s_at | AF081366 | −1.07729 | −1.054648 | potassium inwardly-rectifying channel, subfamily J, member 1 |
| ACC | AF090113_at | AF090113 | 1.373631 | 1.007738 | glutamate receptor interacting protein 2 |
| MPF | AJ006519_at | AJ006519 | 1.775159 | 1.087778 | ether-a-go-go-like potassium channel 1 |
| AMY | AJ007632_s_at | AJ007632 | 1.49322 | 1.553792 | ether-a-go-go-like potassium channel 1 |
| VTA | AJ007632_s_at | AJ007632 | 1.253138 | −1.187813 | 5-hydroxytryptamine (serotonin) receptor 4 |
| VTA | AJ011370_g_at | AJ011370 | 1.015038 | 1.594488 | a disintegrin and metalloproteinase domain 17 |
| VTA | AJ012603cds_at | AJ012603 | 1.00575 | 1.182545 | Bradykinin receptor B1 |
| AMY | AJ132230_at | AJ132230 | −2.171212 | −2.775758 | |
| AMY | D00698_s_at | D00698 | −1.148438 | −4.429688 | glycine receptor, alpha 1 subunit |
| SP | D00833_at | D00833 | −1.811414 | −1.761787 | |
| SP | D00913_g_at | D00913 | 1.053458 | −1.049814 | glutamate receptor, ionotropic, N-methyl D-aspartate 2A |
| ACC | D13211_s_at | D13211 | −1.561062 | −1.097345 | glutamate receptor, ionotropic, NMDA2C |
| VTA | D13212_s_at | D13212 | 1.305048 | 1.262878 | glutamate receptor, ionotropic, NMDA2D |
| VTA | D13213_s_at | D13213 | 1.182025 | 1.003468 | solute carrier family 2, member 2 |
| AMY | D13962_g_at | D13962 | 1.325854 | 1.644394 | chloride channel, nucleotide-sensitive, 1A |
| MPF | D13985_at | D13985 | 1.144137 | 1.275604 | |
| SP | D14478_s_at | D14478 | −1.822938 | 1.245614 | calpain 8 |
| VTA | D14480_at | D14480 | −1.463054 | −1.625616 | sulfotransferase, hydroxysteroid preferring 2 |
| VTA | D14987_f_at | D14987 | 1.332506 | 3.033898 | opioid receptor, mu 1 |
| SP | D16349_at | D16349 | −2.844156 | −1.519481 | retinoblastoma 1 |
| ACC | D25233UTR#1_at | D25233 | 1.740061 | 1.431447 | cadherin 6 |
| SP | D25290_at | D25290 | −1.055077 | −1.512909 | solute carrier family 2, member 5 |
| AMY | D28562_s_at | D28562 | 2.470238 | 2.064677 | |
| SP | D30781_at | D30781 | 1.689826 | 1.685644 | gamma-aminobutyric acid A receptor, rho 2 |
| AMY | D38494_at | D38494 | −1.6 | −1.027273 | gamma-aminobutyric acid A receptor, rho 2 |
| VTA | D38494_at | D38494 | −1.410714 | −2.107143 | |
| SP | D45187_s_at | D45187 | 3.305344 | 1.273529 | |
| AMY | D49395_s_at | D49395 | 2.312404 | 1.375228 | |
| SP | D49395_s_at | D49395 | −2.36646 | −1.701863 | thymoma viral proto-oncogene 3 |
| SP | D49836_at | D49836 | 1.407767 | 1.321185 | thymoma viral proto-oncogene 3 |
| VTA | D49836_at | D49836 | −1.362559 | −1.414692 | GABA receptor rho-3 subunit |
| VTA | D50671_at | D50671 | 1.031746 | −2.061538 | phosphatidylinositol 3-kinase, regulatory subunit, polypeptide 1 |
| AMY | D64045_s_at | D64045 | 1.167504 | 6.453704 | ATP-binding cassette, sub-family C (CFTR/MRP), member 9 |
| SP | D83598_at | D83598 | −1.230915 | −1.18496 | |
| SP | D86039_g_at | D86039 | 1.014658 | 1.011171 | phospholipase D2 |
| AMY | D88672_at | D88672 | −2.093023 | −2.727907 | |
| AMY | D90258_s_at | D90258 | 1.455525 | 1.342944 | |
| AMY | E00988mRNA_s_at | E00988 | −2.423188 | −2.291787 | |
| ACC | E01789cds_s_at | E01789 | 1.016399 | 1.211967 | |
| AMY | E01884cds_s_at | E01884 | 1.086667 | −3.871166 | |
| VTA | E02468cds_s_at | E02468 | −1.536842 | −2.105263 | adrenergic receptor, beta 2 |
| AMY | J03024_at | J03024 | 1.235897 | −2.593361 | adrenergic receptor, beta 2 |
| SP | J03024_at | J03024 | −1.122807 | −2.421053 | muscarinic receptor m2 |
| AMY | J03025_at | J03025 | 1.640288 | 1.002933 | insulin-like growth factor binding protein 2 |
| AMY | J04486_at | J04486 | −2.013337 | −1.484765 | tachikin receptor 3 |
| SP | J05189_at | J05189 | −1.55598 | −1.254453 | cholinergic receptor, nicotinic, alpha polypeptide 5 |
| MPF | J05231_at | J05231 | 1.532199 | 1.152651 | |
| VTA | J05232cds_s_at | J05232 | −1.839442 | −2.813264 | |
| VTA | K01701_at | K01701 | −1.032824 | 1.221992 | protein kinase C, beta 1 |
| ACC | K03486_s_at | K03486 | −1.024357 | 1.087839 | interleukin 10 |
| AMY | L02926_s_at | L02926 | −2.216216 | −1.837838 | |
| ACC | L04739cds_s_at | L04739 | −1.149134 | 1.129425 | |
| VTA | L08492cds_s_at | L08492 | −1.720131 | −1.540098 | |
| AMY | L08494cds_s_at | L08494 | −1.026926 | −1.622289 | |
| ACC | L08495cds_s_at | L08495 | 1.252525 | 1.027406 | |
| AMY | L08497cds_at | L08497 | 1.657866 | 1.393132 | |
| SP | L08497cds_at | L08497 | 1.400592 | −1.249495 | B-cell leukemia/lymphoma 2 |
| SP | L14680_g_at | L14680 | −1.104756 | 1.123692 | glial cell line derived neurotrophic factor |
| VTA | L15305_s_at | L15305 | −1.277778 | 1.830508 | calcium channel, voltage-dependent, L type, alpha 1E subunit |
| SP | L15453_at | L15453 | −1.681587 | −1.351984 | heat shock 70 kD protein 1A |
| VTA | L16764_s_at | L16764 | 1.134927 | 1.136178 | |
| VTA | L19708_at | L19708 | 1.591928 | −1.405634 | prostaglandin-endoperoxide synthase 2 |
| SP | L25925_s_at | L25925 | 1.144598 | 1.725982 | transforming growth factor, beta receptor 1 |
| ACC | L26110_at | L26110 | −1.249791 | −1.382623 | cholinergic receptor, nicotinic, alpha polypeptide 7 |
| SP | L31619_at | L31619 | −2.587209 | −3.19186 | cholinergic receptor, nicotinic, alpha polypeptide 3 |
| AMY | L31621_s_at | L31621 | −3.238806 | −2.331343 | potassium inwardly-rectifying channel, subfamily J, member 9 |
| SP | L77929_at | L77929 | 1.439394 | −1.136842 | tyrosine hydroxylase |
| AMY | M10244_at | M10244 | −1.981224 | −1.32898 | retinol-binding protein 2 |
| AMY | M13949_at | M13949 | 1.372549 | −1.907143 | techykinin 1 |
| VTA | M15191_s_at | M15191 | 1.09605 | 1.498225 | cholinergic receptor, nicotinic, alpha polypetide 4 |
| SP | M15682_at | M15682 | 1.133276 | −1.226859 | cholinergic receptor, nicotinic, alpha polypetide 4 |
| VTA | M15682_at | M15682 | 1.106005 | 1.540364 | calcium/calmodulin-dependent protein kinase II beta subunit |
| AMY | M16112_g_at | M16112 | −1.424992 | −1.16066 | guanine nucleotide binding protein, alpha O |
| AMY | M17526_g_at | M17526 | −1.475709 | −1.198297 | retinol binding protein 1 |
| AMY | M19257_at | M19257 | −1.416257 | −1.711032 | fos-like antigen 1 |
| AMY | M19651_at | M19651 | −1.448276 | −1.42069 | |
| AMY | M20297_at | M20297 | 1.707937 | −1.424721 | sodium channel, voltage-gated, type 2, alpha 1 polypeptide |
| ACC | M22254_at | M22254 | 1.244326 | 1.400619 | potassium voltage-gated channel, isk-related subfamily, member 1 |
| AMY | M22412_at | M22412 | 1.065217 | 2.202247 | interleukin 2 |
| AMY | M22899_at | M22899 | −6.512195 | −3.365854 | interleukin 2 |
| VTA | M22899_at | M22899 | 1.111111 | 2.222222 | muscarinic acetylcholine receptor M5 |
| SP | M22926mRNA_at | M22926 | 1.032065 | 1.487079 | somatostatin |
| AMY | M25890_at | M25890 | 1.003148 | −1.319558 | |
| AMY | M26745cds_s_at | M26745 | −1.680851 | −1.170213 | |
| SP | M26745cds_s_at | M26745 | 1.644737 | 1.644737 | |
| MPF | M27223_at | M27223 | −1.56162 | −1.140845 | insulin-like growth factor 1 receptor |
| SP | M27293_s_at | M27293 | 1.449536 | 1.036299 | cytochrome oxidase subunit VIc |
| AMY | M27466_at | M27466 | 1.06253 | 1.136854 | complement component 3 |
| AMY | M29866_s_at | M29866 | −2.487414 | −3.805492 | complement component 3 |
| MPF | M29866_s_at | M29866 | −1.902098 | −12.11888 | complement component 3 |
| SP | M29866_s_at | M29866 | 1.050366 | 1.023194 | |
| AMY | M30312cds_s_at | M30312 | −2.597107 | −1.280992 | transforming growth factor alpha |
| AMY | M31076_at | M31076 | −1.479487 | −1.378846 | |
| ACC | M31433mRNA#1_at | M31433 | 1.055556 | −1.135711 | |
| MPF | M31433mRNA#1_at | M31433 | −2.049704 | −1.598817 | insulin-like growth factor binding protein 3 |
| AMY | M31837_at | M31837 | −1.792683 | −2.33689 | dopamine receptor 1A |
| AMY | M35077_s_at | M35077 | 1.569207 | 1.637987 | dopamine receptor 1A |
| SP | M35077_s_at | M35077 | −1.239796 | −1.294218 | interleukin 2 receptor, beta chain |
| SP | M55050_at | M55050 | 1.468104 | 1.185776 | glycine receptor, alpha 3 |
| AMY | M55250_at | M55250 | −2.022989 | −3.390805 | neurotrophic tyrosine kinase, receptor, type 2 |
| SP | M55293_at | M55293 | 2.30829 | 1.528302 | calcium channel, voltage-dependent, L type, alpha 1D subunit |
| AMY | M57682_at | M57682 | −3.555556 | −6.981481 | potassium voltage gated channel, Shaw-related subfamily, member 2 |
| SP | M59313_at | M59313 | −10.44444 | −7.388889 | potassium voltage gated channel, Shal-related family, member 2 |
| AMY | M59980_s_at | M59980 | −1.479148 | −1.01712 | adrenergic receptor, alpha 1d |
| AMY | M60654_at | M60654 | −1.742489 | −2.334764 | |
| AMY | M62372cds_s_at | M62372 | −1.082653 | 2.217195 | 5-hydroxytryptamine (serotonin) receptor 2A |
| AMY | M64867_at | M64867 | −1.123096 | −1.085025 | transforming growth factor, beta receptor 3 |
| MPF | M77809_at | M77809 | −1.96 | −2.061667 | transforming growth factor, beta receptor 3 |
| VTA | M80784_s_at | M80784 | 1.416778 | 1.713366 | GABA-alpha receptor gamma-3 subunit |
| VTA | M81142_s_at | M81142 | −3.006623 | −2.721854 | |
| AMY | M82824_s_at | M82824 | −1.192422 | 1.138748 | dopamine receptor 4 |
| VTA | M84009_at | M84009 | −1.88563 | −3.721408 | calcium channel, voltage-dependent, alpha2/delta subunit 1 |
| SP | M86621_at | M86621 | −1.534023 | −1.484962 | |
| AMY | M86742cds_s_at | M86742 | −1.186047 | −1.327696 | ATPase, H+/K+ transporting, nongastric, alpha polypeptide |
| VTA | M90398_at | M90398 | −1.180763 | −1.043118 | adenosine A2B receptor |
| MPF | M91466_at | M91466 | −1.216733 | −1.569721 | |
| AMY | M91595exon_s_at | M91595 | −2.45737 | −1.719755 | |
| AMY | M91599mRNA_g_at | M91599 | 1.097087 | −1.076696 | |
| VTA | M91599mRNA_g_at | M91599 | 1.031686 | 1.11967 | calcium channel, voltage-dependent, N type, alpha 1B subunit |
| ACC | M92905_s_at | M92905 | −1.029584 | −1.236192 | solute carrier family 6 (neurotransmitter transporter, GABA), member 13 |
| AMY | M95762_at | M95762 | −1.825235 | −1.236158 | neurexin 1 |
| ACC | M96375_s_at | M96375 | −1.097785 | 1.356584 | myelin oligodendrocyte glycoprotein |
| AMY | M99485_at | M99485 | 1.41779 | −1.118346 | phosphodiesterase 4B |
| VTA | rc_AA799729_at | AA799729 | 1.051724 | −1.745902 | Rattus norvegicus transcribed sequence with moderate similarity to protein |
| | | | | | sp: O43759 (H. sapiens) |
| AMY | rc_AA799879_at | AA799879 | 1.924747 | 2.227806 | heat shock 70 kD protein 1B |
| VTA | rc_AA818604_s_at | AA818604 | 1.670036 | 1.520845 | Ras-related GTP-binding protein Rab29 |
| SP | rc_AA858977_at | AA858977 | −1.935484 | −2.370968 | Ras-related GTP-binding protein Rab29 |
| VTA | rc_AA858977_at | AA858977 | 5.548673 | 1.583333 | |
| VTA | rc_AA893870_g_at | AA893870 | −1.000483 | −2.301714 | |
| SP | rc_AA894087_at | AA894087 | −1.113038 | −1.12799 | Cbp/p300-interacting transactivator, with Glu/Asp-rich carboxy-terminal |
| | | | | | domain, 2 |
| AMY | rc_AA900476_g_at | AA900476 | −1.278149 | −1.637681 | Cbp/p300-interacting transactivator, with Glu/Asp-rich |
| | | | | | carboxy-terminal domain, 2 |
| SP | rc_AA900476_g_at | AA900476 | −1.241925 | −1.735275 | syntaxin binding protein Munc18-2 |
| VTA | rc_AA964359_s_at | AA964359 | −1.305556 | 1.434263 | heat shock 27 kDa protein 1 |
| VTA | rc_AA998683_g_at | AA998683 | 1.127888 | 1.530233 | leptin receptor |
| ACC | rc_AA998983_at | AA998983 | 1.097345 | 2.384615 | macrophage migration inhibitory factor |
| ACC | rc_AI009801_at | AI009801 | −1.115271 | −1.080384 | glutamine synthetase 1 |
| AMY | rc_AI012265_i_at | AI012265 | 1.452669 | 1.31535 | insulin-like growth factor-binding protein 5 |
| MPF | rc_AI029920_s_at | AI029920 | −1.414406 | 1.01616 | insulin-like growth factor-binding protein 5 |
| VTA | rc_AI029920_s_at | AI029920 | −1.572693 | −1.533197 | brain derived neurotrophic factor |
| MPF | rc_AI030286_s_at | AI030286 | 1.307363 | 1.634904 | nestin |
| AMY | rc_AI030685_s_at | AI030685 | 1.064615 | −1.634393 | solute carrier family 1, member 2 |
| ACC | rc_AI044517_g_at | AI044517 | −1.90683 | −1.959218 | dopa decarboxylase |
| MPF | rc_AI044610_s_at | AI044610 | −1.351994 | −1.56823 | neuronal pentraxin receptor |
| ACC | rc_AI045501_s_at | AI045501 | 1.246668 | 1.160039 | neuronal pentraxin receptor |
| SP | rc_AI045501_s_at | AI045501 | −1.084203 | −1.049448 | |
| VTA | rc_AI137657_at | AI137657 | 1.571429 | −2.194805 | neurabin 1 |
| MPF | rc_AI145444_at | AI145444 | −1.773131 | −1.07858 | |
| VTA | rc_AI146214_at | AI146214 | 1.833937 | 1.620061 | suppressin of tumorigenicity 13 (colon carcinoma) Hsp70-interacting protein |
| AMY | rc_AI171166_at | AI171166 | 1.535167 | −1.03233 | suppressin of tumorigenicity 13 (colon carcinoma) Hsp70-interacting protein |
| SP | rc_AI17116_at | AI171166 | −1.074247 | −1.589304 | heat shock 27 kDa protein 1 |
| VTA | rc_AI176658_s_at | AI176658 | 1.071454 | 1.601921 | guanine nucleotide binding protein, beta 1 |
| VTA | rc_AI227660_s_at | AI227660 | 1.465583 | 1.483917 | Rattus norvegicus hypothetical gene supported by NM_013066 |
| | | | | | (LOC360449), mRNA |
| ACC | rc_AI228850_s_at | AI228850 | −1.268522 | −1.170288 | guanine nucleotide binding protein, beta 1 |
| VTA | rc_AI230404_s_at | AI230404 | 1.511462 | 1.753015 | fos-like antigen 2 |
| AMY | rc_AI230842_at | AI230842 | 1.772426 | 1.778852 | stress activated protein kinase alpha II |
| ACC | rc_AI231354_at | AI231354 | −1.435148 | −1.269825 | stress activated protein kinase alpha II |
| VTA | rc_AI231354_g_at | AI231354 | −1.277972 | 1.141147 | glutathione S-transferase, alpha 1 |
| AMY | rc_AI235747_at | AI235747 | 1.096014 | −1.404132 | |
| AMY | S37461_f_at | S37461 | −1.106762 | −2.124555 | |
| VTA | S37461_f_at | S37461 | −2.819444 | −1.736111 | |
| AMY | S42358_s_at | S42358 | 1.599325 | 1.056807 | |
| SP | S47609_s_at | S47609 | 1.416535 | 1.205197 | |
| VTA | S53987_at | S53987 | 2.082759 | 1.568831 | |
| AMY | S54212_at | S54212 | −1.149194 | 1.04065 | |
| SP | S56481_s_at | S56481 | 1.412098 | 1.313599 | |
| AMY | S59525_s_at | S59525 | −1.747283 | −1.592391 | |
| SP | S59525_s_at | S59525 | −2.636364 | −1.690909 | |
| ACC | S61973_g_at | S61973 | −1.068792 | −1.001844 | |
| SP | S62933_i_at | S62933 | 4.761194 | −1.250784 | |
| SP | S66024_g_at | S66024 | 1.495652 | 1.524505 | |
| AMY | S68944_i_at | S68944 | −1.283297 | 1.64858 | nitric oxide synthase 2, inducible |
| AMY | S71597_s_at | S71597 | 2.052459 | 1.916327 | |
| ACC | S72505_f_at | S72505 | 1.314892 | 1.442016 | |
| VTA | S77863_s_at | S77863 | 1.476378 | −1.024889 | |
| VTA | S78154_at | S78154 | −1.570881 | −1.46798 | |
| ACC | S79676_s_at | S79676 | 1.573875 | 1.454669 | |
| VTA | S79903mRNA_at | S79903 | −1.609047 | −2.927302 | opioid receptor, delta 1 |
| SP | U00475_at | U00475 | −1.6125 | −1.4125 | prostaglandin-endoperoxide synthase 1 |
| ACC | U03388_s_at | U03388 | 1.326597 | −1.057282 | tumor necrosis factor (ligand) superfamily, member 6 |
| VTA | U03470_at | U03470 | −1.829268 | −1.420732 | transforming growth factor, beta 3 |
| AMY | U03491_at | U03491 | −1.270622 | −1.305355 | transforming growth factor, beta 3 |
| SP | U03491_at | U03491 | 1.143872 | 1.05401 | transforming growth factor, beta 3 |
| AMY | U03491_g_at | U03491 | −1.332904 | −1.37311 | glutamate receptor, ionotropic, kainate 4 |
| SP | U08257_at | U08257 | −1.626845 | −1.420664 | glutamate receptor, ionotropic, NMDA2D |
| ACC | U08260_at | U08260 | 1.433073 | 1.233501 | contactin 3 |
| MPF | U11031_at | U11031 | −1.226122 | 1.730226 | |
| VTA | U16359cds_at | U16359 | −1.929929 | −3.06057 | Huntington disease gene homolog |
| VTA | U18650_at | U18650 | 1.112827 | 1.076795 | 5-hydroxytryptamine (serotonin) receptor 4 |
| SP | U20907_at | U20907 | 1.406844 | 4.285714 | 5-hydroxytryptamine (serotonin) receptor 4 |
| VTA | U20907_at | U20907 | 2.306569 | −1.514768 | Eph receptor A7 |
| VTA | U21954_at | U21954 | −1.247423 | −3.020619 | purinergic receptor P2Y, G-protein coupled 1 |
| SP | U22830_at | U22830 | −2.062992 | −1.641732 | purinergic receptor P2Y, G-protein coupled 1 |
| VTA | U22830_at | U22830 | 1.378685 | −1.123355 | potassium inwardly-rectifying channel, subfamily J, member 10 |
| ACC | U27558_at | U27558 | 1.26815 | 1.229943 | glutamate receptor, ionotropic, N-methyl-D-aspartate 3A |
| AMY | U29873_at | U29873 | −1.460894 | −1.061453 | microtubule-associated protein 2 |
| ACC | U30938_at | U30938 | 1.292985 | 1.103875 | calcium channel, voltage-dependent, alpha 1C subunit |
| SP | U31815_s_at | U31815 | −2.529274 | −1.077283 | 5-hydroxytryptamine (serotonin) receptor 2C |
| AMY | U35315_at | U35315 | 1.646552 | 1.281879 | brevican |
| ACC | U37142_at | U37142 | −1.078302 | 1.034517 | |
| ACC | U37147_at | U37147 | −1.411871 | −1.080709 | neuroligin 2 |
| AMY | U41662_at | U41662 | −1.328488 | −1.386628 | complement component 4a |
| AMY | U42719_at | U42719 | −1.814574 | −1.187541 | interleukin 1 receptor-like 2 |
| SP | U49066_at | U49066 | −2.70028 | −1.37535 | caspase 3 |
| SP | U49930_at | U49930 | −1.270548 | −1.40411 | phosphatidylinositol 3-kinase, regulatory subunit, polypeptide 1 |
| SP | U50412_at | U50412 | −1.024514 | −1.131868 | phosphatidylinositol 3-kinase, regulatory subunit, polypeptide 1 |
| VTA | U50412_at | U50412 | 1.172331 | 1.926841 | solute carrier family 7, member 3 |
| AMY | U53927_at | U53927 | −3.135385 | −1.409231 | estrogen receptor 2 |
| SP | U57439_g_at | U57439 | 1.315892 | 1.723785 | metabotropic glutamate receptor 8 |
| AMY | U63288_at | U63288 | 1.121807 | 1.026056 | interleukin 15 |
| SP | U69272_g_at | U69272 | −2.932203 | −4.983051 | potassium intermediate/small conductance calcium-activated |
| | | | | | channel, subfamily N, member 3 |
| ACC | U69884_at | U69884 | −1.192277 | −1.157683 | potassium intermediate/small conductance calcium-activated channel, |
| | | | | | subfamily N, member 3 |
| AMY | U69884_at | U69884 | 1.009804 | −1.724919 | mitogen activated protein kinase 14 |
| VTA | U73142_at | U73142 | 1.10984 | 1.678201 | |
| VTA | U75899mRNA_at | U75899 | −1.191489 | −1.462006 | cyclic nucleotide-gated cation channel |
| SP | U76220_at | U76220 | 1.423913 | 1.984848 | sodium channel, voltage-gated, type 9, alpha polypeptide |
| MPF | U79568_s_at | U79568 | 2.407925 | 1.407357 | interleukin 3 |
| MPF | U81492_s_at | U81492 | −1.439383 | 1.933239 | forkhead box M1 |
| ACC | U83112_at | U83112 | 1.020257 | 1.168978 | guanine nucleotide binding protein, beta 1 |
| VTA | U88324_at | U88324 | 1.353066 | 1.40972 | guanine nucleotide binding protein, beta 1 |
| VTA | U88324_g_at | U88324 | 1.475683 | 1.540453 | calpain 9 (nCL-4) |
| SP | U89514_at | U89514 | 3.348548 | −1.04461 | solute carrier family 1 (high affinity aspartate/glutamate transporter), member 6 |
| VTA | U89608_at | U89608 | 1.745418 | 2.934932 | potassium voltage-gated channel, KQT-like subfamily, member 1 |
| VTA | U92655_at | U92655 | −1.203704 | −1.62963 | CC-chemokine-binding receptor JAB61 |
| AMY | U92803_at | U92803 | 1.297203 | 1.570703 | |
| AMY | X03347cds_g_at | X03347 | −1.100416 | −1.004859 | insulin-like growth factor 1 |
| VTA | X06107_r_at | X06107 | −2.167262 | −2.042173 | RAB3A, member RAS oncogene family |
| ACC | X06889cds_at | X06889 | 1.198613 | 1.195181 | |
| AMY | X06890cds_at | X06890 | 1.53932 | 1.253201 | |
| ACC | X15466cds_at | X15466 | −1.13948 | −1.11323 | |
| AMY | X16002cds_s_at | X16002 | −1.067149 | 1.104727 | insulin-like growth factor 2 |
| SP | X16703_i_at | X16703 | −6.391304 | −7.434783 | insulin-like growth factor 2 |
| VTA | X16703_i_at | X16703 | 1.813084 | 8.818182 | insulin-like growth factor 2 |
| AMY | X16703_r_at | X16703 | 1.102684 | −1.22963 | insulin-like growth factor 2 |
| SP | X16703_r_at | X16703 | 1.921739 | 1.268293 | |
| AMY | X17012mRNA_s_at | X17012 | −2.904584 | −1.653319 | glutamate receptor, ionotropic, AMPA1 (alpha 1) |
| AMY | X17184_at | X17184 | −1.504791 | −1.375871 | |
| ACC | X17621cds_at | X17621 | 1.03678 | 1.010142 | gamma-aminobutyric acid receptor, subunit alpha 3 |
| SP | X51991_at | X51991 | −1.234447 | 1.000514 | complement component 3 |
| VTA | X52477_at | X52477 | −1.399766 | −1.783168 | glycogen synthase kinase 3 beta |
| MPF | X53428cds_s_at | X53428 | 1.492795 | 1.171946 | glycine receptor, alpha 2 subunit |
| AMY | X57281_at | X57281 | 1.424747 | 1.1037 | |
| AMY | X57659_at | X57659 | −1.107223 | 1.046893 | interleukin 10 |
| AMY | X60675_at | X60675 | −1.284543 | −1.350117 | vimentin |
| AMY | X62952_at | X62952 | −2.087663 | −1.376186 | solute carrier family 1, member 3 |
| AMY | X63744_at | X63744 | −1.071485 | 1.825961 | neuronal d4 domain family member |
| AMY | X66022mRNA#3_i_at | X66022 | 1.247934 | 1.365633 | interleukin 4 receptor |
| AMY | X69903_at | X69903 | −1.263048 | −1.103862 | interleukin 4 receptor |
| VTA | X69903_at | X69903 | 1.369725 | 1.74716 | potassium voltage gated channel, shaker related subfamily, beta member 1 |
| ACC | X70662_at | X70662 | 1.142429 | −1.018916 | glycogen synthase kinase 3 beta |
| SP | X73653_at | X73653 | −3.690647 | −4.043165 | inositol 1,4,5-trisphosphate 3-kinase B |
| VTA | X74227cds_at | X74227 | −1.087664 | 1.224293 | cholinergic receptor, nicotinic, delta polypeptide |
| ACC | X74835cds_at | X74835 | −1.029364 | −1.050571 | cholinergic receptor, nicotinic, epsilon polypeptide |
| SP | X74836cds_s_at | X74836 | −1.090328 | 1.597668 | sodium channel, nonvoltage-gated 1 gamma |
| SP | X77933_at | X77933 | −2.274933 | 2.005405 | synuclein, gamma |
| SP | X86789_at | X86789 | −1.34827 | −1.481366 | potassium inwardly-rectifying channel, subfamily J, member 4 |
| VTA | X87635_at | X87635 | −2.175701 | −1.947664 | sodium channel, voltage-gated, type 10, alpha polypeptide |
| VTA | X92184_at | X92184 | 1.299413 | 3.772727 | purinergic receptor P2X, ligand-gated ion channel, 7 |
| ACC | X95882_at | X95882 | 1.623301 | −1.061005 | presenilin-2 |
| ACC | X99267_g_at | X99267 | 1.183964 | 1.023542 | superoxide dismutase 2 |
| SP | Y00497_s_at | Y00497 | 1.112662 | −1.433646 | glutamate receptor, ionotropic, kainate 2 |
| ACC | Z11548_at | Z11548 | 1.485304 | 1.224876 | glutamate receptor, ionotropic, kainate 2 |
| SP | Z11548_at | Z11548 | −1.120169 | −1.130228 | POU domain, class 3, transcription factor 4 |
| AMY | Z11834_at | Z11834 | −1.403939 | −1.075674 | |
| VTA | Z11932cds_g_at | Z11932 | −1.34488 | 1.854749 | platelet derived growth factor, alpha |
| SP | Z14120cds_s_at | Z14120 | 1.404895 | 1.08524 | cAMP responsive element modulator |
| SP | Z15158mRNA_at | Z15158 | −2.92 | −2.38 | |
| AMY | Z38067exon_at | Z38067 | 1.437715 | −1.059766 | met proto-oncogene |
| ACC | Z46374cds_s_at | Z46374 | 1.838655 | 1.404365 | |
| VTA | Z49748exon_at | Z49748 | −1.454545 | −1.325329 | chloride channel 5 |
| MPF | Z56277_i_at | Z56277 | −1.61746 | 1.031097 | potassium voltage-gated channel, subfamily H (eag-related), member 2 |
| VTA | Z96106_at | Z96106 | 1.231948 | 1.795661 | |

1. A method for treating opioid drug addiction of a patient comprising:
administering to the patient, a pharmaceutical agent having a beneficial interaction with any one or more of a) a signaling molecule selected from the group consisting of insulin-like growth factor II, interleukin-3 (IL-3), interleukin-3 beta, fractalkine/chemokine CX3 Cmotif ligand 1, platelet derived growth factor A chain, Neuroligin 3, neuron-specific protein (PEP-19), Synaptamin M;
b) an enzyme selected from the group consisting of catechol-O-methyltransferase, beta-andrenergic receptor kinase, Ras-related GTPase, Ras-related GTPase beta S-100, aromatic L-aminoacid decarboxylase, beta andrenergic receptor kinase, Synaptotagmin VIII, G-protein beta-1 subunit;
c) an ion channel selected from the group consisting of potassium channel beta subunits, sodium channel beta 2 subunit, voltage gated potassium channel Kv3.4, Saw-related subfamily member 2, potassium channel delayed rectifier, potassium inward rectifier 10 (Kir 4.1), calcium channel alpha 1 subunit;
d) a receptor selected from the group consisting of AMPA receptor GluR1, Kainate receptor KA1, Peripheral benzodiazepine receptor, alpha 2-andrenergic receptor, NMDA receptor-like complex glutamate binding protein, GABBA receptor alpha 3 subunit, tumor necrosis factor receptor chain, NMDA receptor subunit 2D, non-processes neurexin1-beta mRNA;
e) a receptor coupling protein;
f) a transporter selected from the group consisting of vescicular inhibitory amino acid transporter and sodium dependent high affinity glutamate transporter, sodium or potassium ion transporting ATPase alpha 2 subunit;
g). a protein from EST AA799879 or AA956149; and
h). a growth, survival, functional, structural protein selected from the group consisting of Bcl-x alpha, signal transducer and activator of transcription 3 (STAT3), Retinoblastoma protein, Nsyndecan (syndecan-3 or Neuroglycan), EST189376, Synaptotagmin VIII, Calcium ion binding protein, Microtubule-associated protein (MAP1A).

2. A method according to claim 1 wherein the beneficial interaction is any one of agonism, antagonism, inhibition, activation, blockage.

3. A method according to claim 2 wherein the beneficial action is agonism, antagonism, mimicry or antimimicry with a signaling molecule.

4. A method according to claim 2 wherein the beneficial action is inhibition, blockage or activation with an enzyme.

5. A method according to claim 2 wherein the beneficial action is blockage or activation with an ion channel.

6. A method according to claim 2 wherein the beneficial action is agonism or antagonism with a receptor.

7. A method according to claim 2 wherein the beneficial action is activation, inhibition, agonism, or antagonism with a receptor coupling protein.

8. A method according to claim 2 wherein the beneficial action is activation or inhibition with a transporter molecule.

9. A method for screening for an interactive pharmaceutical agent comprising:
combining a potential pharmaceutical agent, a label entity and a gene product selected from the group consisting of a signaling molecule, an enzyme, an ion channel, a receptor, a receptor coupling protein, a transporter molecule and a growth/survival/functional/structural protein, wherein the label entity is converted to a detectable label when the candidate chemical entity beneficially interacts with the gene product, and
detecting the presence and/or quantity of detectable label present wherein a positive detection indicates that the potential pharmaceutical agent is an interactive pharmaceutical agent.

10. A method according to claim 9 wherein the combining step is an in vitro process.

11. A method according to claim 9 wherein the combining step is an in vivo process.

12. A method to identify genes comprising comparing expression of an mRNA obtained from a drug addicted animal to expression of the mRNA obtained from a non-drug addicted animal, wherein an increase or decrease in expression of the mRNA obtained from the drug addicted animal relative to expression of the mRNA obtained from the non-drug addicted animal indicates that expression of the mRNA is modulated in response to drug addiction.

13. A method for treating drug addiction of a patient comprising: administering to the patient a pharmaceutical agent having a beneficial interaction with any one or more of syndecan 3, tissue inhibitor of metalloproteinase 3 (TIMP-3), tissue inhibitor of metalloproteinase 2 (TIMP-2), NMDA receptor subunit 2D (NMDA2D), fractalkine or neuroligin 3 so as to treat the drug addiction.

14. A method according to claim 13 wherein the beneficial interaction is any one or more of upregulation, downregulation, agonism, antagonism, inhibition, activation, blockage, mimicry or antimimicry.

15. A method according to claim 14 wherein the beneficial interaction is agonism, antagonism, mimicry or antimimicry with a signaling molecule.

16. A method according to claim 14 wherein the beneficial interaction is inhibition, blockage or activation with an enzyme.

17. A method according to claim 16 wherein the enzyme is a protease or sheddase that cleaves a syndecan 3 and/or fractalkine ectodomain.

18. A method according to claim 14 wherein the beneficial interaction is blockage or activation with an ion channel.

19. A method according to claim 14 wherein the beneficial interaction is agonism or antagonism with a receptor.

20. A method according to claim 14 wherein the beneficial interaction is activation, inhibition, agonism, or antagonism with a receptor coupling protein.

21. A method according to claim 14 wherein the beneficial interaction is activation or inhibition with a transporter molecule.

22. A method for identifying a pharmaceutical agent useful for treating drug addiction comprising:
administering at least one potential pharmaceutical agent and an addicting drug to a nonhuman test mammal;
determining the level of the mRNA and/or protein for at least one of syndecan 3, tissue inhibitor of metalloproteinase 3 (TIMP-3), tissue inhibitor of metalloproteinase 2 (TIMP-2), NMDA receptor subunit 2D (NMDA2D), fractalkine, or neuroligin 3;
and comparing the level of the mRNA and/or protein from the test mammal to the level of the corresponding mRNA and/or protein from a control animal, wherein an alteration in the level of the mRNA and/or protein of at least one of syndecan 3, TIMP-3, TIMP-2, NMDA2D, fractalkine, or neuroligin 3 identifies the pharmaceutical agent as useful for treating drug addiction.

23. A method according to claim 22, wherein the level of the mRNA and/or protein of at least one of syndecan 3, TIMP-3, TIMP-2, NMDA2D, fractalkine, or neuroligin 3 is decreased as compared to the level of the mRNA and/or protein from a control animal.

24. A method according to claim 22, wherein the level of the mRNA and/or protein of at least one of syndecan 3, TIMP-3, TIMP-2, NMDA2D, fractalkine, or neuroligin 3 is increased as compared to the level of the mRNA and/or protein from a control animal.

25. A method according to claim 22, wherein the nonhuman mammal is a rat.

26. An in vitro method for identifying a pharmaceutical agent useful for treating drug addiction comprising:
contacting at least one potential pharmaceutical agent with at least one test cell in vitro;
determining the level of the mRNA and/or protein for at least one of syndecan 3, tissue inhibitor of metalloproteinase 3 (TIMP-3), tissue inhibitor of metalloproteinase 2 (TIMP-2), NMDA receptor subunit 2D (NMDA2D), fractalkine, or neuroligin 3;
and comparing the level of the mRNA and/or protein from the test cell to the level of the corresponding mRNA and/or protein from a control cell, wherein an alteration in the level of the mRNA and/or protein of at least one of syndecan 3, TIMP-3, TIMP-2, NMDA2D, fractalkine, or neuroligin 3 identifies the pharmaceutical agent as useful for treating drug addiction.

27. A method according to claim 26, wherein the level of the mRNA and/or protein of at least one of syndecan 3, TIMP-3, TIMP-2, NMDA2D, fractalkine, or neuroligin 3 is decreased as compared to the level of the mRNA and/or protein from a control cell.

28. A method according to claim 26, wherein the level of the mRNA and/or protein of at least one of syndecan 3, TIMP-3, TIMP-2, NMDA2D, fractalkine, or neuroligin 3 is increased as compared to the level of the mRNA and/or protein from a control cell.

29. A method for screening for a pharmaceutical agent useful for treating drug addiction comprising:
combining a potential pharmaceutical agent, a labeled entity, and a gene product selected from the group consisting of syndecan 3, tissue inhibitor of metalloproteinase 3 (TIMP-3), tissue inhibitor of metalloproteinase 2 (TIMP-2), NMDA receptor subunit 2D (NMDA2D), fractalkine, and neuroligin 3, wherein the labeled entity is converted to a detectable label when the potential pharmaceutical agent interacts so as to alter the presence and/or quantity of the gene product, and
detecting the presence and/or quantity of detectable label present, wherein a positive detection indicates that the potential pharmaceutical agent is a pharmaceutical agent useful for treating drug addiction.

30. A method for identifying a pharmaceutical agent useful for treating drug addiction comprising:
combining a potential pharmaceutical agent, a labeled entity, and a gene product selected from the group consisting of syndecan 3, tissue inhibitor of metalloproteinase 3 (TIMP-3), tissue inhibitor of metalloproteinase 2 (TIMP-2), NMDA receptor subunit 2D (NMDA2D), fractalkine, and neuroligin 3, wherein the labeled entity is converted to a detectable label when the potential pharmaceutical agent interacts so as to alter the presence and/or quantity of the gene product, and
detecting the presence and/or quantity of detectable label present, wherein a positive detection indicates that the potential pharmaceutical agent is a pharmaceutical agent useful for treating drug addiction.

31. A method according to claim 29 wherein the combining step is an in vitro step.

32. A method according to claim 30 wherein the combining step is an in vitro step.

33. A method according to claim 29 wherein the combining step is an in vivo step in a nonhuman mammal.

34. A method according to claim 30 wherein the combining step is an in vivo step in a nonhuman mammal.

35. A method according to claim 33 wherein the nonhuman mammal is a rat.

36. A method according to claim 34 wherein the nonhuman mammal is a rat.
